(19) United States
(12) Patent Application Publication
(10) Pub. No.: US 2018/0282820 A1
(43) Pub. Date: Oct. 4, 2018

(54) MONITORING TREATMENT OR PROGRESSION OF MYELOMA

(71) Applicant: Alfred Health, Prahran (AU)

(72) Inventors: Andrew SPENCER, South Yarra (AU); Sridurga MITHRAPRABHU, Endeavour Hills (AU)

(21) Appl. No.: 15/776,770

(22) PCT Filed: Dec. 2, 2016
(86) PCT No.: PCT/AU2016/051191

§ 371 Date: May 16, 2018

(30) Foreign Application Priority Data
Dec. 3, 2015 (AU) 2015905013
Aug. 1, 2016 (AU) 2016903019

(51) Int. Cl. C12Q 1/6886

(52) U.S. Cl. CPC C12Q 1/6886; C12Q 2600/112; C12Q 2600/106; C12Q 2600/156

(57) ABSTRACT

The present invention relates to methods and kits for diagnosing myeloma, monitoring disease progression or treatment efficacy in an individual having a myeloma. In one aspect, the invention relates to A method for monitoring the response of an individual to treatment for multiple myeloma, the method comprising providing cell-free nucleic acids derived from a sample of peripheral blood from an individual that has undergone treatment for multiple myeloma; assessing the cell-free nucleic acids for a mutation in any one or more nucleotide sequences from a KRAS, NRAS, BRAF and/or TP53 gene; wherein an absence of, or reduction in the number of, mutations in a nucleotide sequence from a KRAS, NRAS, BRAF and/or TP53 gene indicates a response of the individual to treatment for multiple myeloma.

This application claims priority from Australian provisional applications AU 2015905013 and AU 2016903019, the entire disclosures of which are herein incorporated in their entirety.

### FIELD OF THE INVENTION

The present invention relates to methods and kits for determining whether an individual has a myeloma, monitoring progression of a myeloma or the efficacy of treatment for a myeloma.

### BACKGROUND OF THE INVENTION

Multiple myeloma (MM) is an incurable haematological malignancy characterised by multi-focal tumour deposits throughout the bone marrow (BM). Karyotypic instability and numeric chromosome abnormalities are present in virtually all MM. Primary translocations involving the immunoglobin (IgH) gene and FGFR3/MMSET, CCND1, CCND3, or MAF occur during the disease pathogenesis and secondary translocation involving the MYC gene occurs during disease progression. Treatment of MM has witnessed significant progress with the implementation of proteasome inhibitors and immunomodulatory agents, however, the disease remains incurable with cells acquiring resistance to systemic therapies through accumulation of mutations that are often not present during the initial stages of the disease. Resistance to therapy is often mediated through genetic evolution of the MM cells, with the more resistant clones possessing a growth and survival advantage. Current practice for diagnosis and prediction of prognosis is to perform sequential BM biopsies but the genetic information (GI) obtained from biopsies is confounded by the known inter and intra-clonal heterogeneity of the tumour(s).

There exists a need for improved or alternative methods for determining diagnosis, prediction of prognosis of multiple myeloma and/or monitoring efficacy of treatment.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a method for monitoring the response of an individual to treatment for multiple myeloma, the method comprising
providing cell-free nucleic acids derived from a sample of peripheral blood from an individual that has undergone treatment for multiple myeloma;
assessing the cell-free nucleic acids for a mutation in any one or more nucleotide sequences from a KRAS, NRAS, BRAF and/or TP53 gene;
   wherein an absence of, or reduction in the number of, mutations in a nucleotide sequence from a KRAS, NRAS, BRAF and/or TP53 gene indicates a response of the individual to treatment for multiple myeloma; or wherein the presence of, or increase in the number of mutations in a nucleotide sequence from a KRAS, NRAS, BRAF and/or TP53 gene indicates a non-response of the individual to treatment for multiple myeloma.

The present invention provides a method for monitoring the response of an individual to treatment for multiple myeloma, the method comprising
providing cell-free nucleic acids derived from a sample of peripheral blood from an individual that has undergone treatment for multiple myeloma;
providing nucleic acids from bone marrow mononuclear cells of the individual;
assessing the cell-free nucleic acids for a mutation in any one or more nucleotide sequences from a KRAS, NRAS, BRAF and/or TP53 gene;
assessing the nucleic acids from bone marrow mononuclear cells for a mutation in any one or more nucleotide sequences from a KRAS, NRAS, BRAF and/or TP53 gene;

wherein an absence of, or reduction in the number of, mutations in a nucleotide sequence from a KRAS, NRAS, BRAF and/or TP53 gene in either or both the cell-free nucleic acids or the nucleic acids from bone marrow mononuclear cells indicates a response of the individual to treatment for multiple myeloma; or wherein an presence of, or increase in the number of, mutations in a nucleotide sequence from a KRAS, NRAS, BRAF and/or TP53 gene in either or both the cell-free nucleic acids or the nucleic acids from bone marrow mononuclear cells indicates a non-response of the individual to treatment for multiple myeloma.

The present invention provides a method for monitoring the response of an individual to treatment for multiple myeloma, the method comprising:
assessing a test sample of peripheral blood from an individual that has undergone treatment for multiple myeloma, thereby forming a test sample profile;
comparing the test sample profile with a control profile to identify whether there is a difference in the level of cell-free nucleic acids as between the test sample profile and the control profile, the control profile containing data on the level of cell-free nucleic acids in peripheral blood of individuals without multiple myeloma;
determining that the individual has a response to treatment for multiple myeloma, where the level of cell-free nucleic acids in the test sample profile is the same as the control profile.

The present invention provides a method for monitoring the response of an individual to treatment for multiple myeloma, the method comprising:
assessing a test sample of peripheral blood from an individual that has undergone treatment for multiple myeloma, thereby forming a test sample profile;
comparing the test sample profile with a control profile to identify whether there is a difference in the level of cell-free nucleic acid as between the test sample profile and the control profile, the control profile containing data on the level of cell-free nucleic acid in peripheral blood of the individual before treatment commenced;
determining that the individual has a response to treatment for multiple myeloma, where the level of cell-free nucleic acid in the test sample profile is lower than the control profile.

The present invention provides a method for monitoring the response of an individual to treatment for multiple myeloma, the method comprising:
assessing a test sample of peripheral blood from an individual that has undergone treatment for multiple myeloma, thereby forming a test sample profile;
comparing the test sample profile with a control profile to identify whether there is a difference in the level of cell-free nucleic acid as between the test sample profile and the control profile, the control profile containing data on the level of cell-free nucleic acid in peripheral blood of the individual before treatment commenced;
determining that the individual has a response to treatment for multiple myeloma, where the level of cell-free nucleic acid in the test sample profile is lower than in the control profile; or
determining that the individual has not had a response to treatment for multiple myeloma, where the level of cell-free nucleic acid in the test sample profile is the same or higher than in the control profile.

The present invention provides a method for monitoring the response of an individual to treatment for multiple myeloma, the method comprising:
providing a test sample of peripheral blood from an individual that has undergone treatment for multiple myeloma;
assessing the test sample for the level of cell-free nucleic acid, thereby forming a test sample profile;
providing a control profile containing data on the level of cell-free nucleic acid in peripheral blood of individuals without multiple myeloma;
comparing the test sample profile with the control profile to identify whether there is a difference in the level of cell-free nucleic acid as between the test sample profile and the control profile;
determining that the individual has a response to treatment for multiple myeloma, where the level of cell-free nucleic acid in the test sample profile is the same as the control profile.

The present invention provides a method for monitoring the response of an individual to treatment for multiple myeloma, the method comprising:
providing a test sample of peripheral blood from an individual that has undergone treatment for multiple myeloma;
assessing the test sample for the level of cell-free nucleic acid, thereby forming a test sample profile;
providing a control profile containing data on the level of cell-free nucleic acid in peripheral blood of individuals having multiple myeloma;
comparing the test sample profile with the control profile to identify whether there is a difference in the level of cell-free nucleic acid as between the test sample profile and the control profile;
determining that the individual has a response to treatment for multiple myeloma, where the level of cell-free nucleic acid in the test sample profile is lower than in the control profile; or
determining that the individual has no response to treatment for multiple myeloma, where the level of cell-free nucleic acid in the test sample profile is the same of higher than in the control profile.

In any aspect of the invention described herein, the cell-free nucleic acid is cell-free DNA. In any aspect of the invention described herein, the cell-free nucleic acid is cell-free tumour-derived DNA.

The present invention also provides a method for monitoring the response of an individual to treatment for multiple myeloma, the method comprising
providing cell-free nucleic acids derived from a sample of peripheral blood from an individual that has undergone treatment for multiple myeloma;
assessing the cell-free nucleic acids for a mutation in any one or more nucleotide sequences from a KRAS, NRAS, BRAF and/or TP53 gene;

wherein an absence of mutations in a nucleotide sequence from a KRAS, NRAS, BRAF and/or TP53 gene indicates a response of the individual to treatment for multiple myeloma.

The present invention also provides a method for monitoring the response of an individual to treatment for multiple myeloma, the method comprising
providing cell-free nucleic acids derived from a sample of peripheral blood from an individual that has undergone treatment for multiple myeloma;
providing nucleic acids from bone marrow mononuclear cells of the individual;
assessing the cell-free nucleic acids for a mutation in any one or more nucleotide sequences from a KRAS, NRAS, BRAF and/or TP53 gene;
assessing the nucleic acids from bone marrow mononuclear cells for a mutation in any one or more nucleotide sequences from a KRAS, NRAS, BRAF and/or TP53 gene;

wherein an absence of mutation in a nucleotide sequence from a KRAS, NRAS, BRAF and/or TP53 gene in either or both the cell-free nucleic acids or the nucleic acids from bone marrow mononuclear cells indicates a response of the individual to treatment for multiple myeloma.

The present invention provides a method for monitoring the response of an individual to treatment for multiple myeloma, the method comprising:
assessing the cell-free nucleic acids of peripheral blood from an individual that has undergone treatment for multiple myeloma for a mutation in any one or more nucleotide sequences from a KRAS, NRAS, BRAF and/or TP53 gene thereby forming a test sample profile;
comparing the test sample profile with a control profile to identify whether there is a difference in the number of mutation or level of cell-free nucleic acids that contain at least one mutation as between the test sample profile and the control profile, the control profile containing data on the level of cell-free DNA in peripheral blood of the individual before treatment commenced;
determining that the individual has responded to treatment for multiple myeloma wherein the number of mutation or level of cell-free nucleic acid that contain at least one mutation in the test sample profile is lower than the control profile. Alternatively, the determining step that the individual has not responded to treatment for multiple myeloma is wherein the number of mutation or level of cell-free nucleic acid that contain at least one mutation in the test sample profile is the same or higher than the control profile.

The present invention also provides a method for monitoring the response of an individual to treatment for multiple myeloma, the method comprising
providing an individual that has undergone treatment for multiple myeloma, that has been diagnosed as having multiple myeloma, or that has been identified as having advanced disease, according to a method of the invention as described herein;
assessing the cell-free nucleic acids for a mutation in any one or more nucleotide sequences from a KRAS, NRAS, BRAF and/or TP53 gene;

wherein an absence of, or reduction in the number of, mutations in a nucleotide sequence from a KRAS, NRAS, BRAF and/or TP53 gene indicates a response of the individual to treatment for multiple myeloma.

In any aspect of the invention, a step of providing a test sample of peripheral blood may involve obtaining a peripheral blood sample directly from the individual to be diagnosed or monitored.

In any aspect of the invention, assessing the cell-free nucleic acids for a mutation may include determining the number of, or fractional abundance of, transcripts having that mutation.

Preferably, the step of assessing the test sample for the level of cell-free nucleic acid or number of mutations in cell-free nucleic acid, typically DNA, includes extracting cell-free nucleic acid from the peripheral blood and discarding all components of the peripheral blood except for the cell-free nucleic acid.

In any aspect of the invention above, there further comprises the step of administering one or more drugs to treat the individual. Preferably, the treatment includes administering a drug or drugs which is/are different to that previously administered to the patient, such that the overall treatment of the individual for multiple myeloma is modified. In some embodiments, the drug or drugs that were previously administered to the patient is/are supplemented with one or more additional drugs. In alternative embodiments, the drug or drugs that were previously administered is/are replaced with one or more alternative drugs.

Preferably, the drug administered is a therapy known to a skilled person including Dexamethasone, Cyclophosphamide, Thalidomide, Lenalinomide, Etopside, Cisplatin, Ixazomib, Bortezomib, Vemurafinib, Rigosertib, Trametinib, Panobinostat, Azacytidine, Pembrolizumab, Nivolumumab, Durvalumab or autologous stem cell transplant (ASCT). The treatment may include one or more drugs, or any combination of two or more drugs including in the following combinations: Dexamethasone, Cyclophosphamide, Etoposide and Cisplatin (DCEP); Dexamethasone, Cyclophosphamide, Etoposide, Cisplatin and Thalidomide (T-DCEP); Lenalidomide and Dexamethasone (Rd), Ixazomib-cyclophosphamide-dexamethasone (ICd); or Bortezomib, Cyclophosphamide and Dexamethasone (VCD). The treatment may include combinations of DCEP, T-DCEP, Rd, Icd or VCD in combination with additional drugs.

In any aspect of the invention above, the step of administering a drug to treat the individual occurs wherein the determining step identifies the patient as failing to respond to treatment or identifies the patient as having a mutational load higher in cell-free nucleic acids derived from a sample of peripheral blood or circulating tumour free nucleic acids than in corresponding bone marrow derived nucleic acids.

The present invention provides a method for determining a treatment regimen for an individual who has multiple myeloma, the method comprising:
providing an individual that is receiving or has undergone treatment for multiple myeloma, or that has been identified as having advanced disease, according to a method of the invention as described herein;
providing cell-free nucleic acids derived from a sample of peripheral blood from the individual;
assessing the cell-free nucleic acids for a mutation in any one or more nucleotide sequences from a KRAS, NRAS, BRAF and/or TP53 gene;

wherein detecting mutations selected from the group consisting of any one of KRAS, NRAS. BRAF and/or TP53 mutations determines that the treatment regimen comprises administration of a drug which specifically targets the KRAS, NRAS, BRAF and/or TP53 pathways.

It will be understood that where an individual is determined to have more than one mutation, the treatment may include more than one drug such that each mutation is specifically targeted.

The present invention also includes the step of determining to cease administration of a particular drug and commence an alternative treatment where it is determined that the mutations of the individual are not responsive to the current treatment protocol. In addition, the present invention includes the step of determining to maintain administration of a drug which targets a specific mutation, and supplementing the treatment protocol by the addition of one or more drugs which target different mutations in the individual.

The present invention provides a method for monitoring the disease progression of an individual having multiple myeloma, the method comprising:
providing a test sample of peripheral blood from an individual for whom the progression of multiple myeloma is to be determined;
assessing the test sample for the level of circulating tumour free nucleic acids or number of mutations in tumour free nucleic acids in a KRAS, NRAS, BRAF and/or TP53 gene, thereby forming a test sample profile;
providing a comparative profile containing data on level of circulating tumour free nucleic acids or number of mutations in tumour free nucleic acids in a KRAS, NRAS, BRAF and/or TP53 gene of the same individual at a previous time;
comparing the test sample profile with the comparative profile to identify whether there is a difference in the level of level of circulating tumour free nucleic acids or number of mutations in tumour free nucleic acids as between the test sample profile and the comparative profile;
determining that the disease in the individual has progressed where the level of circulating tumour free nucleic acids, or numbers of mutations in tumour free nucleic acids in the test sample profile is higher than the comparative profile. Alternatively, determining that the disease is the individual has not progressed where the level of circulating tumour free nucleic acids, or numbers of mutations in tumour free nucleic acids in the test sample profile is the same or lower than the comparative profile.

Preferably, the comparative profile from the same individual at a previous time is from at least 1, 2, 3, 4, 5, 6, 7, 8, 9, 10, 11, 12 or 24 months prior to conducting the method of the invention.

Preferably, a step of providing a test sample of peripheral blood involves obtaining a peripheral blood sample directly from the individual to be diagnosed.

Preferably, the step of assessing the test sample for the existence of, the level of, or numbers of mutations in, circulating tumour free nucleic acids includes extracting cell-free DNA from the peripheral blood and discarding all components of the peripheral blood except for the cell-free DNA.

The present invention provides a method for monitoring the progression of disease in an individual having multiple myeloma, the method comprising:
providing cell-free nucleic acids derived from a sample of peripheral blood from an individual for whom disease progression is to be determined;
assessing the cell-free nucleic acids for one or more mutations in a nucleotide sequence from a TP53 gene;

wherein detection of one or more mutations in TP53 diagnoses the individual as having progressed to advanced disease. Preferably, the mutations in TP53 encode any one or more mutations listed in FIG. 10.

The present invention provides a method for monitoring the progression of disease in an individual having multiple myeloma, the method comprising:
providing cell-free nucleic acids derived from a sample of peripheral blood and bone marrow mononuclear cells from an individual for whom a diagnosis of multiple myeloma is to be determined;
assessing the cell-free and bone marrow derived nucleic acids for mutations in any one or more of the KRAS, NRAS, BRAF or TP53;

wherein detection of greater than 3 TP53 mutations diagnoses the individual as having progressed to advanced disease. Preferably, the mutations in TP53 encode any one or more mutations listed in FIG. 10.

The present invention provides a method for diagnosing an individual as having multiple myeloma, or at risk of developing same, the method comprising:
assessing a test sample of peripheral blood from an individual for whom a diagnosis of multiple myeloma is to be determined for circulating tumour free nucleic acids,

wherein a determining the presence of circulating tumour free nucleic acids diagnoses that the individual has multiple myeloma, or is at risk of developing same. Preferably, circulating tumour free nucleic acids are cell-free nucleic acids in which at least one mutation is present in a nucleotide sequence from a KRAS, NRAS, BRAF and/or TP53 gene. Preferably, the mutation is any one or more that encodes a mutation listed in FIG. 10.

The present invention provides a method for diagnosing an individual as having multiple myeloma, or at risk of developing same, the method comprising:
providing a test sample of peripheral blood from an individual for whom a diagnosis of multiple myeloma is to be determined;
assessing the test sample for circulating tumour free nucleic acids,

wherein a detection of circulating tumour free nucleic acids diagnoses that the individual has multiple myeloma, or is at risk of developing same. Preferably, circulating tumour free nucleic acids are cell-free nucleic acids in which at least one mutation is present in a nucleotide sequence from a KRAS, NRAS, BRAF and/or TP53 gene. Preferably, the mutation encodes any one or more of the mutations listed in FIG. 10.

The present invention provides a method for diagnosing an individual as having multiple myeloma, or at risk of developing same, the method comprising:
providing cell-free nucleic acids derived from a sample of peripheral blood from an individual for whom a diagnosis of multiple myeloma is to be determined;
assessing the cell-free nucleic acids for a mutation in any one or more nucleotide sequences from a KRAS, NRAS, BRAF and/or TP53 gene;

wherein detection of a mutation in any one or more of the KRAS, NRAS, BRAF or TP53 diagnoses that the individual has multiple myeloma, or is at risk of developing same. Preferably, the method comprises a step of obtaining a peripheral blood sample from the individual from which cell-free nucleic acids are extracted.

In any aspect of the invention, the mutation detected in the nucleic acid encodes a mutation selected from any one or more of the group consisting of those shown in FIG. 10. More preferably, the mutation is selected from any one or more of KRAS G12D, KRAS G12C, KRAS G12V, KRAS G12S, KRAS G12R, KRAS G12A, KRAS G13C, NRAS Q61K, NRAS Q61H_1, NRAS G13D, NRAS Q61H, NRAS Q61L, NRAS G13R, BRAF V600E, and TP53 R273H. More preferably, the mutation is selected from KRAS G12S, KRAS G12R and NRAS Q61L.

The present invention provides a method for diagnosing an individual as having multiple myeloma, or at risk of developing same, the method comprising:
providing a plurality of probes designed to detect mutations known to be associated with multiple myeloma;
contacting cell-free nucleic acids derived from a peripheral blood sample with the plurality of probes under conditions suitable to allow binding of a probe to a cell-free nucleic acid; and
detecting the binding of the probes to the cell-free nucleic acid.

The present invention provides a method for diagnosing an individual as having multiple myeloma, or at risk of developing same, the method comprising:
providing a plurality of probes designed to detect one or more mutations in a nucleic acid encoding a mutation selected from the group consisting of those shown in FIG. 10;
contacting cell-free nucleic acids derived from a peripheral blood sample with the plurality of probes under conditions suitable to allow binding of a probe to a cell-free nucleic acid; and
detecting the binding of the probes to the cell-free nucleic acid.

The present invention provides a method for diagnosing an individual as having multiple myeloma, or at risk of developing same, the method comprising:
providing cell-free nucleic acids derived from a sample of peripheral blood from an individual for whom a diagnosis of multiple myeloma is to be determined;
assessing the cell-free nucleic acids for a mutation in any one or more sequences from a KRAS, NRAS, BRAF and/or TP53 gene;
providing nucleic acids from bone marrow mononuclear cells from the individual;
assessing the nucleic acids from bone marrow mononuclear cells for a mutation in any one or more nucleotide sequences from a KRAS, NRAS, BRAF and/or TP53 gene;

wherein detection of mutations in both the cell-free nucleic acids and the nucleic acids from bone marrow diagnoses the individual as having multiple myeloma.

The present invention provides a method for diagnosing an individual as having multiple myeloma, or at risk of developing same, the method comprising:
assessing a test sample of peripheral blood from an individual for whom a diagnosis of multiple myeloma is to be determined for the level of cell-free DNA, thereby forming a test sample profile;
comparing the test sample profile with the control profile to identify whether there is a difference in the level of cell-free DNA as between the test sample profile and the control profile, the control profile containing data on the level of cell-free DNA in peripheral blood of individuals without multiple myeloma;
determining that the individual has multiple myeloma, or is at risk of developing same, where the level of cell-free DNA in the test sample profile is higher than the control profile.

The present invention provides a method for diagnosing an individual as having multiple myeloma, or at risk of developing same, the method comprising:
providing a test sample of peripheral blood from an individual for whom a diagnosis of multiple myeloma is to be determined;
assessing the test sample for the level of cell-free DNA, thereby forming a test sample profile;
providing a control profile containing data on the level of cell-free DNA in peripheral blood of individuals without multiple myeloma;
comparing the test sample profile with the control profile to identify whether there is a difference in the level of cell-free DNA as between the test sample profile and the control profile;
determining that the individual has multiple myeloma, or is at risk of developing same, where the level of cell-free DNA in the test sample profile is higher than the control profile.

The present invention provides a method for diagnosing an individual as having multiple myeloma, or at risk of developing same, the method comprising:
providing a test sample of peripheral blood from an individual for whom a diagnosis of multiple myeloma is to be determined;
assessing the test sample for the level of cell-free DNA, thereby forming a test sample profile;
providing a comparative profile containing data on the level of cell-free DNA in peripheral blood of the same individual at a previous time;
comparing the test sample profile with the comparative profile to identify whether there is a difference in the level of cell-free DNA as between the test sample profile and the comparative profile;
determining that the individual has multiple myeloma, or is at risk of developing same, where the level of cell-free DNA in the test sample profile is higher than the comparative profile.

Any aspect of the invention described above can be used to identify an individual for to treatment with a modality that targets the Ras-MAPK pathway, preferably the modality is an inhibitor of the Ras-MAPK pathway. For example, identification of a mutation in a gene that encodes for a product involved in the Ras-MAPK pathway identifies the individual as likely to benefit from treatment with an inhibitor of the Ras-MAP K pathway.

In any aspect of the invention, the mutations in a nucleotide sequence from a KRAS, NRAS, BRAF and/or TP53 gene encode a mutation in the amino acid sequence selected from the group consisting of those listed in FIG. 10.

In any aspect of the invention described herein, there further comprises the step of administering a drug to treat the individual diagnosed as having multiple myeloma, active disease or advanced disease. A drug to treat multiple myeloma may be any one typically used for treatment including those described herein.

In any aspect of the invention described herein, assessing for mutations comprises comparing a nucleotide sequence comprising all, or part, of a KRAS, NRAS, BRAF or TP53 gene from the individual with a nucleotide sequence comprising all, or part, of a KRAS, NRAS, BRAF or TP53 gene from a control individual or individuals (e.g. derived from one or more individuals without multiple myeloma or with newly diagnosed, non-advanced disease as the case may be).

The present invention also provides a kit for use in diagnosing an individual as having multiple myeloma, or at risk of developing same, or for use in monitoring the progression or stage of disease or monitoring treatment efficacy, the kit comprising:
a means for detecting any one or more mutations in a nucleotide sequence from a KRAS, NRAS, BRAF and/or TP53 gene that encode mutations elected from the group consisting of those listed in FIG. 10;
reagents for isolating or extracting cell-free nucleic acids from a peripheral blood sample of an individual.

Preferably, the kit also comprises the nucleotide sequence of a KRAS, NRAS, BRAF and/or TP53 gene from an individual that does not have multiple myeloma.

Preferably, the kit also comprises the wildtype sequence of a KRAS, NRAS, BRAF and/or TP53 gene at the positions where a mutation identified in a patient with multiple myeloma has been detected. Typically, the position where a mutation identified in a patient with multiple myeloma is listed in FIG. 10.

Preferably, the kit also comprises written instructions for use of the kit in a method of the invention as described herein.

Preferably, the means for detecting one or more mutations is one or more nucleic acid probes or primers to either hybridize with a sequence including the mutation or amplify a sequence including the mutation. It is preferred that the probes are oligonucleotide probes, which bind to their target sites within the sequence of a KRAS, NRAS, BRAF and/or TP53 gene by way of complementary base-pairing. For the avoidance of doubt, in the context of the present invention, the definition of an oligonucleotide probe does not include the full length KRAS, NRAS, BRAF and/or TP53 gene (or the complement thereof).

The present invention also provides a multiple myeloma detection system comprising a plurality of probes fixed to a solid support for use, or when used, in a method as described herein. Preferably, the probes are designed to detect one or more mutations selected from the group listed in FIG. 10.

In any aspect herein, the bone marrow mononuclear cells may be from a bone marrow biopsy.

The present invention also provides a method of treating an individual having multiple myeloma comprising administering a drug to treat the individual, wherein the individual is diagnosed as having multiple myeloma by any method of the invention described herein. Preferably, the drug administered is a therapy known to a skilled person including Dexamethasone, Cyclophosphamide, Thalidomide, Lenalinomide, Etopside, Cisplatin, Ixazomib, Bortezomib, Vemurafinib, Rigosertib, Trametinib, Panobinostat, Azacytidine, Pembrolizumab, Nivolumumab, Durvalumab or autologous stem cell transplant (ASCT). The treatment may include one or more drugs, or any combination of two or more drugs including in the following combinations: Dexamethasone, Cyclophosphamide, Etoposide and Cisplatin (DCEP); Dexamethasone, Cyclophosphamide, Etoposide, Cisplatin and Thalidomide (T-DCEP); Lenalidomide and Dexamethasone (Rd), Ixazomib-cyclophosphamide-dexamethasone (ICd); or Bortezomib, Cyclophosphamide and Dexamethasone (VCD). The treatment may include combinations of DCEP, T-DCEP, Rd, Icd or VCD in combination with additional drugs.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Cell-free DNA (cfDNA) amounts are significantly higher in plasma from patients with multiple myeloma (MM). Column graph indicates the amount of cfDNA in ng recovered from 1 ml of plasma (PL) from MM patients (n=37) and normal volunteers (NV) (n=21). The amounts in MM are significantly higher as assessed by Mann-Whitney t-test utilising GraphPad Prism V6 for statistical analysis and indicates a significance of p=0.0085.

FIG. 2: cfDNA amounts correlate to disease stage. Column graph indicates the amount of cfDNA in ng recovered from 1 ml of PL in NV, and in MM patients with active and stable disease. The levels of cfDNA in patients with active disease are significantly higher than in NV (p=0.0067) compared using Mann-Whitney t-test.

FIG. 3: cfDNA amounts do not correlate with paraprotein, serum free light chain (SFLC) or the bone marrow (BM) MM cell proportions. Correlation plot indicates that the amount of cfDNA does not correlate with the amounts of paraprotein, SFLC and BM MM cell proportions. Pearson's correlation coefficient analysis was performed to determine r-value for correlation using GraphPad Prism V6f.

FIG. 4: Distribution of mutations in paired BM and PL samples of MM patients. Column graph represents the number of mutations and proportions of KRAS, NRAS, TP53 and BRAF present in BM and PL samples.

FIG. 5: Distribution of mutations in relapsed/refractory (RR) and new diagnosis (ND) patients. Column graph indicates the number of mutations found in BM and PL within each patient. Ten of the 18 48RR patients demonstrated mutations detectable only in PL consistent with mutationally disparate disease distant from the site of BM biopsy (the top section of the column in RR1RR2, 4, 10, 12, 13, 14, 15, 28, 35 and 8 and 11, and ND 13).

FIG. 6: Mutational abundance (MA) in BM and PL of samples. The dot-plots are a representation of the MA of mutations present in BM, PL, or both BM and PL. The median levels of MA are shown. The median levels of MA in the BM are significantly higher than in PL (p=0.014) for mutations detected in both compartments. In the mutations found in both BM and PL, the median MA in BM was significantly higher than the median MA in mutations found only in the BM (p<0.0001). The MA of PL only mutations was significantly lower than the MA of PL mutations detected in both the BM and PL (p=0.003). All analyses were performed using Mann-Whitney t-test.

FIG. 7: Distribution of type of mutations (A) All mutations detected in the BM and/or PL of the 48 patients. NRAS Q61K was the most prevalent. (B) KRAS mutations detected (C) NRAS mutations detected (D) TP53 mutations detected, and (E) BRAF mutations detected.

FIG. 8: MM has predominantly KRAS mutations. Proportion of KRAS, NRAS, BRAF and TP53 mutations detected in (A) BM only (B) PL only (C) Both BM and PL.

FIG. 9: Distribution of mutations in ND and RR patients. Column graph represents the number and type of mutations present within each RR and ND patient. One or more RAS mutations were present in over 69% of the patients.

FIG. 10: List of KRAS, NRAS, BRAF and TP53 mutations in the OMD panel.

FIG. 11: Summary of mutations detected in bone marrow (BM), peripheral blood (PB) samples or both in patients.

FIG. 12: Sequential tracking of mutant clones in PL of patients #1-3.

(A): Line graph represents the FA of mutant clones by ddPCR in Patient #1. PL was collected at 1, 2, 3, 5, 8 and 10 months post-diagnosis. Serum kappa free-light chains (Kappa LC) levels are shown on the right Y-axis with overt disease progression evident at month 10. A marked increase in mutant clone KRAS G12D FA but not TP53 R273H plotted on the left Y-axis coincided with serological progression while on oral azacytidine, revlimid and dexamethasone (Rd) therapy.

(B): Line graph represents the FA of mutant clones KRAS G12V and KRAS G12S (on the left Y-axis) in sequential PL collected at months 1, 2, 6, 12, 15 and 17 while on revlimid and dexamethasone. Lambda light chains (LC) and paraprotein (right Y-axis) declined at month 12 followed by an increase at month 15 and 17. Levels of KRAS G12V coincided with Lambda LC increase during therapy.

(C): Line graph represents the FA of mutant KRAS G12C in sequential PL collected at months 1, 4 and 13 post—allograft (Allo) (left Y-axis). FA levels coincided with Kappa light chains (LC) and were present at detectable levels consistent with stable disease at month 4 and 13 post-allo shown in the right Y-axis.

Sequential tracking of mutant clones in patient PL. Line graph represents the FA of mutant clones by ddPCR in Patient #3. PL was collected at 1, 2, 3, 5, 8 and 10 months post-diagnosis (shown as red asterisk). Serum kappa free-light chains (Kappa LC) levels are shown with overt disease progression evident at month 10. A marked increase in mutant clone KRAS G12D FA but not TP53 R273H coincided with serological progression while on oral azacytidine, revlimid and dexamethasone (Rd) therapy.

FIG. 13: Sequential tracking of mutant clones in PL of patients #4, #5, #6, and #7.

(A) Line graph represents FA of PL-only mutations KRAS G13C in patient #4 in PL collected at months 1, 4 and 7 of newly diagnosed patient on panobinostat therapy. No significant changes were detected in both Lambda light chains (LC) and paraprotein levels between months 4 and 7; however, KRAS G13C levels had a sharp increase between months 4 and 7 consistent with disease relapse (left Y-axis).

(B) Detection of PL mutations during therapy for Patient #5. Line graph represents the FA of mutant clones NRAS Q61K, KRAS Q61H_1 and BRAF V600E in patient#5 PL collected at day 1, 10, 20 and 90 while on oral azacytidine, revlimid and dexamethasone (Rd). FA levels decreased at 10 days of treatment while Kappa light chains (LC) decrease was detected only from day 20.

(C) Line graph represents the FA of 4 mutant clones (left Y-axis) and Lambda LC (right Y-axis) in sequential PL of relapsed patients collected at months 1, 13 and 24 during therapy. Patient #6 relapsed on revlimid and dexamethasone with increase in levels of two mutant clones KRAS G12V and KRAS G12A at month 13 coinciding with Lambda LC, however, TP53 R273H and NRAS G13R FA were found to decrease. A switch to Ixazomib, cyclophosphamide and dexamethasone (Cd) at month 13 decreased levels of KRAS G12A and KRAS G12V with increasing levels of NRAS G13R suggesting differential response of mutant clones to treatment.

(D) Line graph represents the FA of mutant clones by ddPCR in a non-secretory patient, Patient #7. PL was collected at 1, 3, 13, 17 and 19 months post-diagnosis. The proportion of BM MM cells is shown with an increasing FA of 4 clones coinciding with BM relapse at month 13, only 9 months post-autologous stem cell transplantations (ASCT). At month 19 a BM response to VCD was evident but with an increasing FA of the NRAS G13D clone. The patient succumbed to refractory progressive disease shortly afterwards.

FIG. 14: Validation of OnTarget™ Mutation Detection platform (OMD) results using ddPCR. Table summarises the BM and PL samples that were checked for specific mutations using ddPCR for the presence (√) or absence (X) of mutations.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

The present inventors have determined methods of diagnosing multiple myeloma and various stages of multiple myeloma disease progression by detecting cell-free DNA in peripheral blood. The present invention therefore provides significant advantages including that it is possible to monitor disease progression and response to treatment via a non-invasive method (blood sampling vs bone marrow biopsy) and the detection of mutational status via cell-free DNA provides a more comprehensive picture of the genetic signature of tumours than a tissue biopsy of a single site. These advantages allow more robust diagnosis and allows specific treatments to be aligned with the genetic alterations present in the disease. More specifically, the inventors have shown that where conventional methods for monitoring disease progression may indicate that treatment has been successful, the methods of the present invention enable a more accurate assessment of the progression of the disease including more precise monitoring of disease kinetics. This type of insight enables the clinician to provide a more personalised treatment, wherein specific molecular pathways can be targeted by adapting the treatment protocol in response to the mutational status that is determined for the individual, including as the mutational status of the individual changes over the course of the disease. Further, the methods of the present invention enable earlier intervention in circumstances where one treatment approach is no longer effective, facilitating the adaptation of the treatment protocol to reflect changes in the mutational status of the individual, as the disease progresses.

Nucleic acids are released into the plasma and serum through cellular apoptosis, necrosis and spontaneous release of DNA/RNA-lipoprotein complexes amongst other sources. Circulating cell-free tumor-derived DNA (ctDNA) contains a representation of the entire tumour genome with DNA sourced from multiple independent tumours. Whole genome or exome sequencing of this ctDNA can be utilised to identify mutations associated with acquired resistance to cancer therapy without the need to perform sequential biopsies of the tumour. It has also become evident that secondary mutations are more readily detectable in the plasma than via re-biopsy of the primary tumour, due to the high false-negative rate associated with the latter, validating the utility of plasma-based analysis for the characterisation of target oncogenes and the identification of mutations that are acquired during disease progression. Therefore, available data would suggest that analysis of circulating nucleic acids provides a potentially more comprehensive picture of the genetic landscape of tumour(s) than tissue biopsy of a single site. The inventors have obtained results described herein that show a more comprehensive picture of the genetic landscape of individual MM patients is to analyse circulating cell-free nucleic acids, i.e. cell free-DNA and RNA (cfDNA and cfRNA, respectively), derived from the peripheral blood (PB), as this contains a representation of the entire tumour genome and transcriptome that may arise from multiple independent tumours.

A ‘cell-free nucleic acid’, or “cfDNA” as used herein, is a nucleic acid, preferably DNA (genomic or mitochondrial), that has been released or otherwise escaped from a cell into blood or other body fluid in which the cell resides. The extraction or isolation of cell-free nucleic acid (e.g. DNA) from a body fluid, such as peripheral blood, does not involve the rupture of any cells present in the body fluid. Cell-free DNA may be DNA isolated from a body fluid in which all or substantially all particulate material in the fluid, such as cells or cell debris, has been removed.

Where cell-free nucleic acid is derived from a tumour (i.e., nucleic acid that originates from a tumour and is released into the blood or other body fluid), the term cell-free tumor-derived DNA or ctDNA can be used.

Cell-free nucleic acids, such as DNA, may be extracted from peripheral blood samples using techniques including e.g. Lo et al, U.S. Pat. No. 6,258,540; Huang et al, Methods Mol. Biol, 444: 203-208 (2008); and the like, which are incorporated herein by reference. By way of nonlimiting example, peripheral blood may be collected in EDTA tubes, after which it may be fractionated into plasma, white blood cell, and red blood cell components by centrifugation. DNA present in the cell-free plasma fraction (e.g. from 0.5 to 2.0 mL) may be extracted using a QIAamp DNA Blood Mini Kit (Qiagen, Valencia, Calif.), or like kit, in accordance with the manufacturer's protocol.

Unless the context states otherwise, circulating cell-free tumor-derived nucleic acid and circulating tumour free nucleic acids are used interchangeably, as are cell-free tumor-derived DNA and circulating tumour free DNA.

The present invention can be used to diagnose, monitor disease progression or treatment efficacy in an individual. The present invention can be used to characterise the mutational status or landscape of an individual with myeloma, including to characterise changes in mutational status over the course of the disease in the individual and/or in response to various treatment approaches.

Monitoring disease progression or treatment efficacy may be of an individual having any type of multiple myeloma including smouldering or indolent multiple myeloma, active multiple myeloma, multiple solitary plasmacytomas, extramedullary plasmacytoma, secretory, non-secretory, IgG lambda or kappa light chain (LC) types. The most common immunoglobulins (Ig) made by myeloma cells in multiple myeloma are IgG, IgA and IgM, less commonly, IgD or IgE is involved.

Aspects of the present invention, such as monitoring disease progression or treatment efficacy, may be particularly useful in individuals where no conventional peripheral blood biomarker (e.g. no paraprotein, or other marker described herein including the Examples, or known in the art) is detectable.

The methods of the present invention typically include a comparison of nucleic acids from the individual (sometimes referred to as a “test sample”) with nucleic acids in a control profile.

In some instances, the ‘control profile’ may include the level of cell free nucleic acid, preferably cell-free DNA, from a peripheral blood sample of an individual or individuals that do not have any clinically or biochemically detectable multiple myeloma. In such instances, the peripheral blood sample of an individual or individuals that do not have any clinically or biochemically detectable multiple myeloma is herein referred to as the ‘control sample’. The ‘control profile’ may be derived from an individual that, but for an absence of multiple myeloma, is generally the same or very similar to the individual selected for determination of whether they have multiple myeloma. The measurement of the level of cell-free DNA in the control sample from the peripheral blood of the individual or individuals for deriving the control profile is generally done using the same assay format that is used for measurement of the cell-free DNA in the test sample.

It will be appreciated that the control profile may also be derived from the same individual from which the test sample is taken, but at a different time-point, for example, a year or several years earlier. As such, the control profile may also include the level of cell-free nucleic acid from the individual before the individual received treatment for multiple myeloma, or at an earlier stage during the treatment of multiple myeloma, Such a control profile thereby forms a baseline or basal level profile of the level of cell-free DNA in the individual, against which the test sample may be compared.

In addition to providing a measure of the level of cell-free nucleic acid, the control profile may also provide information on the presence or absence of specific mutations, as described herein, those mutations being detected in cell-free nucleic acids from an individual.

A control profile for measuring disease progression or monitoring treatment efficacy may be generated from the same individual from which the test sample is taken, but at a different time-point, for example, a year or several years earlier. Such a control profile thereby forms a baseline or basal level profile in the individual of the (a) level of circulating tumour free nucleic acid, (b) number of mutations in the circulating tumour free nucleic acid, or (c) proportion of circulating tumour free nucleic acid that contains at least one or more mutations.

In the present specification failure of treatment includes progression of disease while receiving a treatment (e.g. chemotherapy) regimen without experiencing any transient improvement, no objective response after receiving one or more cycles of a treatment regimen or a limited response with subsequent progression while receiving a treatment regimen. Myeloma that is not responsive to therapy may also be termed ‘Refractory multiple myeloma’. Refractory myeloma may occur in patients who never see a response from their treatment therapies or it may occur in patients who do initially respond to treatment, but do not respond to treatment after relapse.

In the present specification ‘relapse’ means, unless otherwise specified, the return of signs and symptoms of cancer after a period of improvement.

As used herein ‘advanced disease’ includes individuals that have relapsed and/or have refractory multiple myeloma.

The words ‘treat’ or ‘treatment’ or ‘response to treatment’ refers to therapeutic treatment wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Treatment can also mean prolonging survival as compared to expected survival if not receiving treatment. Treatment may not necessarily result in the complete clearance of a disease or disorder but may reduce or minimise complications and side effects of infection and the progression of a disease or disorder.

Although the invention finds application in humans, the invention is also useful for therapeutic veterinary purposes. The invention is useful for domestic or farm animals such as cattle, sheep, horses and poultry; for companion animals such as cats and dogs; and for zoo animals.

The present invention also provides a mutational status of the RAS/MAPK pathway in an individual which can then be used to identify individuals who may be treatable by a therapeutic modality that targets the RAS/MAPK pathway such as trametinib, rigosertib, cobimetinib, selumetinib, sorafenib or vemurafenib.

The present invention includes monitoring the efficacy of a treatment for multiple myeloma, wherein the treatment includes but is not limited to administration of any one or more of: Dexamethasone, Cyclophosphamide, Thalidomide, Lenalinomide, Etopside, Cisplatin, Ixazomib, Bortezomib, Vemurafinib, Rigosertib, Trametinib, Panobinostat, Azacytidine, Pembrolizumab, Nivolumumab, Durvalumab or autologous stem cell transplant (ASCT).

The treatment may include one or more drugs, or any combination of two or more drugs including in the following combinations: Dexamethasone, Cyclophosphamide, Etoposide and Cisplatin (DCEP); Dexamethasone, Cyclophosphamide, Etoposide, Cisplatin and Thalidomide (T-DCEP); Azacytidine and Lenalidomide (Rd), Ixazomib-cyclophosphamide-dexamethasone (ICd); or Bortezomib, Cyclophosphamide and Dexamethasone (VCD). The treatment may include combinations of DCEP, T-DCEP, Rd, Icd or VCD in combination with additional drugs.

The present invention also includes adapting or modifying a treatment for multiple myeloma based on the results of determining or monitoring the mutational status of an individual receiving treatment for multiple myeloma. The adaption or modification may include removing a particular drug or drugs from the treatment protocol and replacing the drug with one or more alternative drugs. Alternatively, the adaptation or modification may include supplementing the existing treatment with additional drugs.

In any embodiment, the replacement or supplemental treatment includes administering any one or more of Dexamethasone, Cyclophosphamide, Thalidomide, Lenalinomide, Etopside, Cisplatin, Bortezomib, Cobimetinib, Ixazomib, Rigosertib, Selumetinib, Sorafenib Trametinib, Vemurafinib, Panobinostat, Azacytidine, Pembrolizumab, Nivolumumab, Durvalumab or autologous stem cell transplant (ASCT). The replacement or supplemental treatment may also include administering any one or more of the combinations of: Dexamethasone, Cyclophosphamide, Etoposide and Cisplatin (DCEP); Dexamethasone, Cyclophosphamide, Etoposide, Cisplatin and Thalidomide (T-DCEP); Lenalidomide and Dexamethanasone (Rd), Ixazomib-cyclophosphamide-dexamethasone (ICd); or Bortezomib, Cyclophosphamide and Dexamethasone (VCD). The treatment may include combinations of DCEP, T-DCEP, Rd, Icd or VCD in combination with additional drugs.

By monitoring the progression and change of mutational status of the individual using the methods of the present invention, the clinician or practitioner is able to make informed decisions relating to the treatment approach adopted for any one individual. For example, in certain embodiments, it may be determined that specific mutant clones identified in a MM patient do not respond to a first treatment, but do respond to a second treatment while other clones identified in the individual, respond to the first but not the second treatment. Thus, by monitoring the response of mutant clones to various treatment approaches using the methods of the present invention, it is also possible to tailor an approach which combines two or more treatments, each targeting different subsets of clones in the individual.

The following are some scenarios illustrating the utility of the present invention in adapting treatment for MM over the course of the disease:
an individual receives treatment with a combination of lenalinomide (Revlimid) and dexamethasone for several months. Over the course of the treatment, levels of paraprotein and Lambda LC gradually decrease, as does the abundance of clones in plasma having the KRAS G12S mutation. After 15 months of treatment, the fractional abundance of KRAS G12V clones in plasma dramatically increases at a rate which exceeds only a modest increase in the amount of paraprotein and Lambda LC. The results indicate that a change in the treatment protocol is required so as to specifically target KRAS G12V clones. Given the efficacy of lenalinomide-dexamethasone in targeting KRAS G21S clones, supplementation of the existing treatment protocol with an additional drug that targets the RAS pathway is recommended;
an individual receives treatment with the combination of azacytidine and Rd (lenalinomide and dexamethasone). After several months of treatment, fractional abundance of KRAS G12D clones in plasma increases dramatically, indicating a modification in the treatment protocol to target the RAS/MAPK pathway is required;
an individual receives treatment with ASCT two months after diagnosis with MM. In the months following treatment, abundance of KRAS G31C clones decreases. Following switching of treatment to Panabinostat, the fractional abundance of KRAS G13C clones increases indicating that this drug does not successfully target these clones and an alternative or supplemental treatment is required;
an individual receives treatment with the combination Rd (lenalinomide and dexamethasone). Over the course of the treatment, fractional abundance of TP53 R273H and RNAS G13R clones decreased, indicating that these clones responded to treatment with Rd. However, abundance of KRAS G12V and G12A clones increased indicating that these clones were not responsive to the initial treatment. Replacement of Rd treatment with Ixazomib, cyclophosphamide and dexamethasone (Icd) showed that that KRAS G12V and G12A clones responded to treatment but that fractional abundance of RNAS G13R clones increased. ICd treatment was then supplemented with Rd treatment, thus targeting KRAS G12V and G12A and RNAS G13R clones.;
an individual receives treatment with Bortezomib, Cyclophosphamide and Dexamethasone (VCD) followed by ASCT. The fractional abundance of various clones in plasma decreases after the initial treatment and increases only slightly over the course of the following months. Dexamethasone, Cyclophosphamide, Etoposide, Cisplatin and Thalidomide (T-DCEP) treatment is commenced in response to an increase in bone marrow MM. The T-DCEP treatment has no effect and there is a subsequent increase in fractional abundance of G13D clones and NRAS Q61K clones. Subsequent switching to VCD treatment successfully targets the bone marrow MM and NRAS Q61K clones but does not target the NRAS G13D clones indicating that supplemental treatment with a drug which targets this clone is required.

Each of the above scenarios indicates that monitoring the progression of disease in accordance with the methods of the present invention enables the replacement or supplementation of existing treatments, so as to specifically target clones which have increased functional abundance in plasma as the disease progresses.

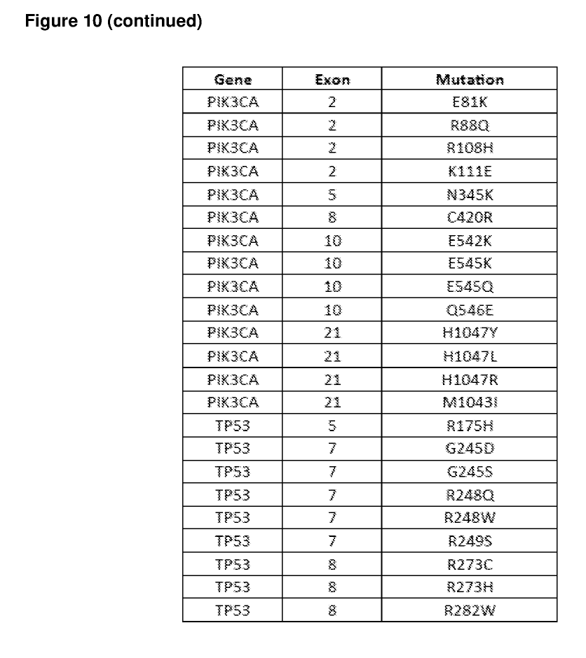

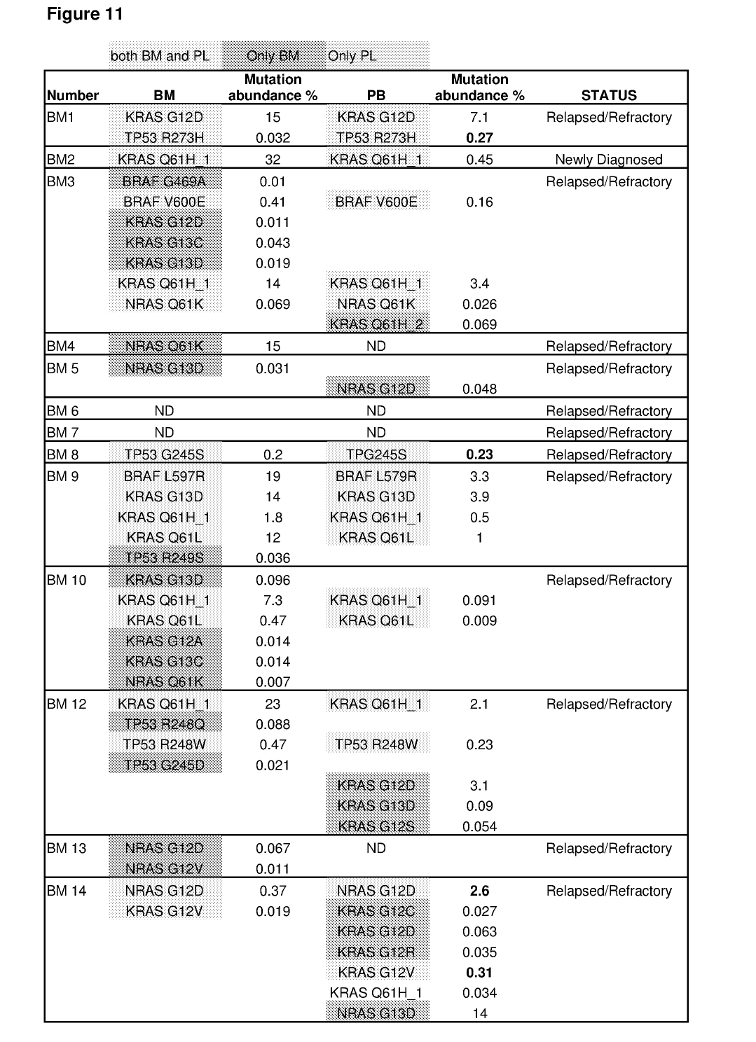

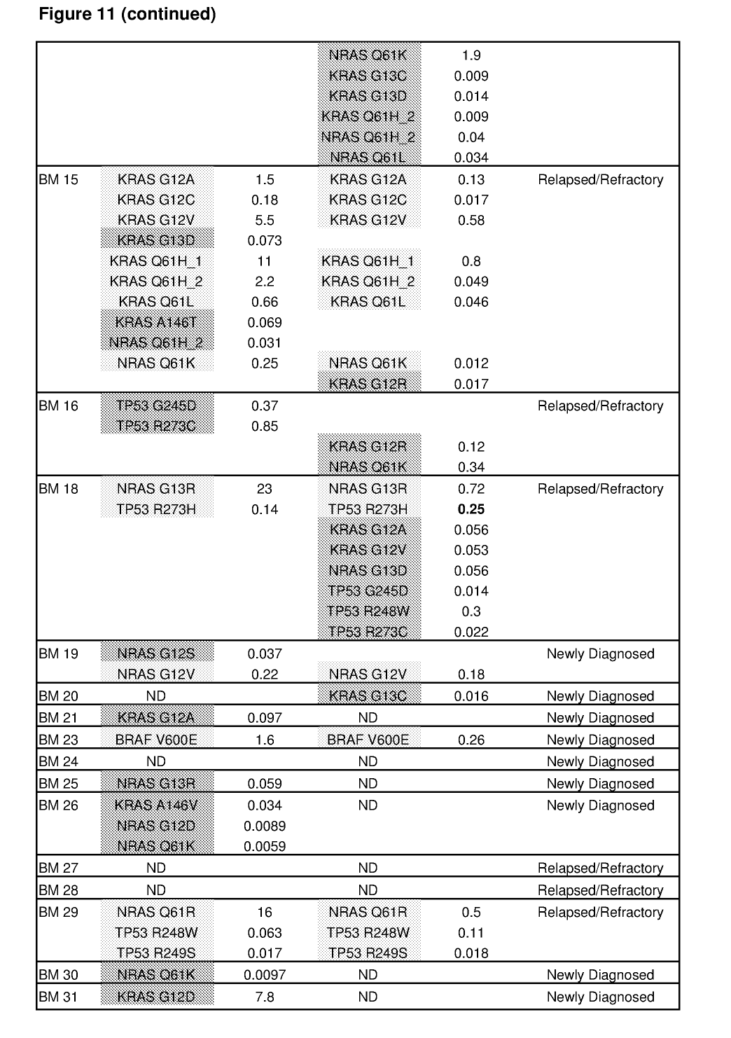

Mutations described in FIGS. 10 and 11 and referred to herein as useful in the invention are shown as amino acid mutations in a particular protein. For example, KRAS G12D, is refers to a mutation in the gene encoding, KRAS which causes a change at position 12 from glycine (G) which appears in the wildtype, normal protein to an aspartate (D). Any mutation in the nucleic acid that causes the amino acid mutation in FIG. 10 is contemplated herein. The numbering of all amino acid mutations corresponds to the position in wildtype human amino acid sequence from the given protein. However, the amino acid residue number may be different in another animal so the invention contemplates mutations that are equivalent to those shown in FIG. 10 in an ortholog or paralog from a human or other animal described herein. The nucleotide sequences of the KRAS, NRAS, BRAF or TP53 genes are known and can be accessed by any known database such as the GenBank database, for example, human KRAS by accession number NM_004985.3, human NRAS by accession number NM_002524.4, human BRAF by accession number NM_004333.4, and human TP53 by accession number NM_000546.5.

GTPase KRAS also known as V-Ki-ras2 Kirsten rat sarcoma viral oncogene homolog and KRAS, is a protein that in humans is encoded by the KRAS gene. KRAS may be referred to as KRAS; C-K-RAS; CFC2; K-RAS2A; K-RAS2B; K-RAS4A; K-RAS4B; KI-RAS; KRAS1; KRAS2; NS; NS3; RASK2.

NRAS is an enzyme that in humans is encoded by the NRAS gene. NRAS may be referred to as NRAS; ALPS4; CMNS; N-ras; NCMS; NRAS1; NS6.

BRAF is a human gene that makes a protein called B-Raf. The gene is also referred to as proto-oncogene B-Raf and v-Raf murine sarcoma viral oncogene homolog B, while the protein is more formally known as serine/threonine-protein kinase B-Raf. BRAF may be referred to as BRAF; B-RAF1; BRAF1; NS7; RAFB1.

Tumor protein p53, also known as p53, cellular tumor antigen p53 (UniProt name), phosphoprotein p53, tumor suppressor p53, antigen NY-CO-13, or transformation-related protein 53 (TRP53), is any isoform of a protein encoded by homologous genes in various organisms, such as TP53 (humans) and Trp53 (mice). TP53 may be referred to as TP53; BCC7; LFS1; P53; TRP53.

As used herein, the term ‘nucleic acid’ refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. Alternatively, it can be a single-stranded nucleic acid not derived from any double-stranded DNA. Suitable nucleic acid molecules are DNA, including genomic DNA or cDNA. Other suitable nucleic acid molecules are RNA, including mRNA.

The term ‘isolated’ or ‘partially purified’ as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered ‘isolated’.

As used herein, a ‘portion’ of a nucleic acid molecule refers to contiguous set of nucleotides comprised by that molecule. A portion can comprise all or only a subset of the nucleotides comprised by the molecule. A portion can be double-stranded or single-stranded.

As used herein, ‘amplified product’, ‘amplification product’, or ‘amplicon’ refers to oligonucleotides resulting from an amplification reaction that are copies of a portion of a particular target nucleic acid template strand and/or its complementary sequence, which correspond in nucleotide sequence to the template nucleic acid sequence and/or its complementary sequence. An amplification product can further comprise sequence specific to the primers and which flanks sequence which is a portion of the target nucleic acid and/or its complement. An amplified product, as described herein will generally be double-stranded DNA, although reference can be made to individual strands thereof.

In any method of the invention described herein, assessing or determining in a sample of an amount, level, presence of, or mutations in (a) circulating cell-free tumor-derived nucleic acid or circulating tumour free nucleic acids, or (b) cell-free nucleic acids, may be by any method as described herein, for example a form of PCR, microarray, sequencing etc.

An amount of a nucleic acid may be quantified using any method described herein, or for example, the polymerase chain reaction (PCR) or, specifically quantitative polymerase chain reaction (QPCR) or droplet digital polymerase chain reaction (DDPCR). QPCR is a technique based on the polymerase chain reaction, and is used to amplify and simultaneously quantify a targeted nucleic acid molecule. QPCR allows for both detection and quantification (as absolute number of copies or relative amount when normalized to DNA input or additional normalizing genes) of a specific sequence in a DNA sample. The procedure follows the general principle of polymerase chain reaction, with the additional feature that the amplified DNA is quantified as it accumulates in the reaction in real time after each amplification cycle. QPCR is described, for example, in Kurnit et al. (U.S. Pat. No. 6,033,854), Wang et al. (U.S. Pat. Nos. 5,567,583 and 5,348,853), Ma et al. (The Journal of American Science, 2(3), 2006), Heid et al. (Genome Research 986-994, 1996), Sambrook and Russell (Quantitative PCR, Cold Spring Harbor Protocols, 2006), and Higuchi (U.S. Pat. Nos. 6,171,785 and 5,994,056). The contents of these are incorporated by reference herein in their entirety.

One example is the OnTarget™ Mutation Detection (OMD) platform (Boreal Genomics) described in the Examples.

Any high-throughput technique for sequencing nucleic acids can be used in the method of the invention to determine the amount, level or mutations in cell-free nucleic acids or cell-free tumour nucleic acids. A variety of sequencing technologies are available with such capacity, which are commercially available, Illumina, Inc. (San Diego, Calif.); Life Technologies, Inc. (Carlsbad, Calif.). In some embodiments, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In another aspect, such methods comprise amplifying the isolated molecules either before or after they are spatially isolated on a solid surface. Prior amplification may comprise emulsion-based amplification, such as emulsion PCR, or rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010); and further in the following references: U.S. Pat. Nos. 6,090,592; 6,300,070; 7,115,400; and EP0972081B1; which are incorporated by reference. Whole exome sequencing is described in the Examples.

The mutations as described herein may be detected using probes, preferably oligo nucleotides. Probes are designed to bind to the target gene sequence based on a selection of desired parameters, using conventional software. It is preferred that the binding conditions are such that a high level of specificity is provided—ie. binding occurs under “stringent conditions”. In general, stringent conditions are selected to be about 5° C. lower than the thermal melting point.

(Tm) for the specific sequence at a defined ionic strength and pH. The Tm is the temperature (under defined ionic strength and pH) at which 50% of the target sequence binds to a perfectly matched probe. In this regard, the Tm of probes of the present invention, at a salt concentration of about 0.02M or less at pH 7, is preferably above 40° C. and preferably below 70° C., more preferably about 53° C. Premixed binding solutions are available (eg. EXPRESSHYB Hybridisation Solution from CLONTECH Laboratories, Inc.), and binding can be performed according to the manufacturer's instructions. Alternatively, one of a skill in the art can devise variations of these binding conditions.

Following binding, washing under stringent (preferably highly stringent) conditions removes unbound nucleic acid molecules. Typical stringent washing conditions include washing in a solution of 0.5-2×SSC with 0.1% SDS at 55-65° C. Typical highly stringent washing conditions include washing in a solution of 0.1-0.2×SSC with 0.1% SDS at 55-65° C. A skilled person can readily devise equivalent conditions for example, by substituting SSPE for the SSC in the wash solution.

Apart from the stringency of the hybridization conditions, hybridization specificities may be affected by a variety of probe design factors, including the overall sequence similarity, the distribution and positions of mismatching bases, and the amount of free energy of the DNA duplexes formed by the probe and target sequences.

The ‘complement’ of a nucleic acid sequence binds via complementary basepairing to said nucleic acid sequence. A non-coding (anti-sense) nucleic acid strand is also known as a “complementary strand”, because it binds via complementary base-pairing to a coding (sense) strand.

Thus, in one aspect, the probe binds to a target sequence within the coding (sense) strand of the nucleotide sequence of a KRAS, NRAS, BRAF and/or TP53 gene containing any one or more of the mutations listed in FIG. 10. Alternatively, in another aspect, the probe binds to a target sequence within the complementary, non-coding (anti-sense) strand of the nucleotide sequence of a KRAS, NRAS, BRAF and/or TP53 gene containing any one or more of the mutations listed in FIG. 10.

In one aspect, the probe may be immobilised onto a support or platform. Immobilising the probe provides a physical location for the probe, and may serve to fix the probe at a desired location and/or facilitate recovery or separation of probe.

The support may be a rigid solid support made from, for example, glass or plastic, or else the support may be a membrane, such as nylon or nitrocellulose membrane. 3D matrices are suitable supports for use with the present invention—eg. polyacrylamide or PEG gels.

In one embodiment, the support may be in the form of one or more beads or microspheres, for example in the form of a liquid bead microarray. Suitable beads or microspheres are available commercially (eg. Luminex Corp., Austin, Tex.). The surfaces of the beads may be carboxylated for attachment of DNA. The beads or microspheres may be uniquely identified, thereby enabling sorting according to their unique features (for example, by bead size or colour, or a unique label), In one aspect, the beads/microspheres are internally dyed with fluorophores (eg. red and/or infrared fluorophores) and can be distinguished from each other by virtue of their different fluorescent intensity.

In one aspect, prior to contacting the nucleotide sequence of a KRAS, NRAS, BRAF and/or TP53 gene containing any one or more of the mutations listed in FIG. 10 with said oligonucleotide probe, the method further comprises the step of amplifying a portion of the KRAS, NRAS, BRAF and/or TP53 gene, or the complement thereof, thereby generating an amplicon.

It may be desirable to amplify the target nucleic acid if the sample is small and/or comprises a heterogeneous collection of DNA sequences.

Amplification may be carried out by methods known in the art, and is preferably carried out by PCR. A skilled person would be able to determine suitable conditions for promoting amplification of a nucleic acid sequence.

Thus, in one aspect, amplification is carried out using a pair of sequence specific primers, wherein said primers bind to target sites in the KRAS, NRAS, BRAF and/or TP53 gene, or the complement thereof, by complementary basepairing. In the presence of a suitable DNA polymerase and DNA precursors (dATP, dCTP, dGTP and dTTP), the primers are extended, thereby initiating the synthesis of new nucleic acid strands which are complementary to the individual strands of the target nucleic acid. The primers thereby drive amplification of a portion of the KRAS, NRAS, BRAF and/or TP53 gene, or the complement thereof, thereby generating an amplicon. This amplicon comprises the target sequence to which the probe binds, or may be directly sequenced to identified the presence of one or more mutations as described herein.

For the avoidance of doubt, in the context of the present invention, the definition of an oligonucleotide primer does not include the full length KRAS, NRAS, BRAF and/or TP53 gene (or complement thereof).

The primer pair comprises forward and reverse oligonucleotide primers. A forward primer is one that binds to the complementary, non-coding (antisense) strand of the target nucleic acid and a reverse primer is one that binds to the corresponding coding (sense) strand of the target nucleic acid. As used herein, target nucleic acid is a nucleic acid that comprises a nucleotide sequence of a KRAS, NRAS, BRAF and/or TP53 gene in which the presence of a mutation, preferably a mutation listed in FIG. 10, is to be determined.

Primers of the present invention are designed to bind to the target gene sequence based on the selection of desired parameters, using conventional software, such as Primer Express (Applied Biosystems). In this regard, it is preferred that the binding conditions are such that a high level of specificity is provided. The melting temperature (Tm) of the primers is preferably in excess of 50° C. and is most preferably about 60° C. A primer of the present invention preferably binds to target nucleic acid but is preferably screened to minimise self-complementarity and dimer formation (primer-to-primer binding).

The forward and reverse oligonucleotide primers are typically 1 to 40 nucleotides long. It is an advantage to use shorter primers, as this enables faster annealing to target nucleic acid.

Preferably the forward primer is at least 10 nucleotides long, more preferably at least 15 nucleotides long, more preferably at least 18 nucleotides long, most preferably at least 20 nucleotides long, and the forward primer is preferably up to 35 nucleotides long, more preferably up to 30 nucleotides long, more preferably up to 28 nucleotides long, most preferably up to 25 nucleotides long. In one embodiment, the forward primer is about 20-21 nucleotides long.

Preferably the reverse primers are at least 10 nucleotides long, more preferably at least 15 nucleotides long, more preferably at least 20 nucleotides long, most preferably at least 25 nucleotides long, and the reverse primers are preferably up to 35 nucleotides long, more preferably up to 30 nucleotides long, most preferably up to 28 nucleotides long. In one embodiment, the reverse primer is about 26 nucleotides long.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nl, to a few hundred μl, e.g. 200 μl. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences.

Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, p2-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

### EXAMPLES

### Example 1

Peripheral Blood (PB) Collection and Processing:

PB samples (30 mls) were obtained from normal volunteers (NV) or MM patients using 10 ml Streck Cell-Free DNA BCT tubes or 10 ml EDTA tubes following informed consent as per the Alfred Human Ethics Committee. Immediately upon sample collection, the tubes were inverted to mix the blood with the preservative in the collection tube preventing the release of DNA from blood cells during sample processing and storage (Das K, et al. (2014) Molecular diagnosis & therapy 18(6):647-653; Qin J, Williams T L, & Fernando M R (2013) BMC research notes 6:380). Plasma (PL) was separated from PB through centrifugation at 820×g for 10 minutes (mins). Supernatant was collected without disturbing the cellular layer and centrifuged again at 16,000×g for 10 mins to remove any residual cellular debris. The supernatant was collected and stored at −80° C. in 1 ml aliquots for long-term storage until isolation of cfDNA from plasma.

Cell-Free DNA Extraction:

Frozen plasma samples were used for cfDNA extraction using the QIAamp circulating nucleic acid kit (Qiagen, Germany) according to manufacturers' instructions. An average of 6 ml of plasma was used for cfDNA extractions. Subsequently, plasma ctDNA was quantified with a QUBIT Fluorometer and high sensitivity DNA detection kits (Life Technologies, Australia). DNA yield were measured using the Qubit 2.0 Fluorometer (Life Technologies). The maximum input volume utilised for the QUBIT assay was 5 μl. The extracted DNA was stored at −80° C. until further processing.

Ficoll Isolation of Bone Marrow Mononuclear Cells, Determination of MM Cell Proportion and Isolation of CD138+ MM Cells

Coincident with PB sampling BM from MM patients or NV was obtained following written informed consent as per Alfred Hospital Human Ethics Committee-approved protocol. Ficoll Paque Plus (GE Healthcare, Rydalmere, NSW, Australia) was utilized to isolate buffy layers containing bone marrow mononuclear cells (BMMNC) as per manufacturer's guidelines. Red blood cells were removed using red blood cell lysis buffer (10 mmol/L KHCO₃, 150 mmol/L NH₄Cl and 0.1 mmol/L EDTA, pH 8.0) for 5 minutes at 37° C. followed by removal of any lysis buffer by washing with sterile phosphate buffered saline (PBS). Cells were then cultured overnight in RPMI-1640 media supplemented with 10% FCS and 2 mM L-glutamine. Proportion of MM or normal plasma cells (PC) in BMMNC isolated from each patient was determined through flow cytometric enumeration of CD138, CD38 and CD45 staining. Briefly, cells were stained with CD45-FITC (Becton Dickinson (BD) Biosciences, North Ryde, NSW, Australia), CD38-PerCP (BD Biosciences) and CD138-PE (Miltenyi Biotec, Macquarie Park, NSW, Australia) for 20 minutes at room temperature, washed and resuspended in 300 μL of buffer (0.5% FCS/PBS). Samples were acquired on a BD FACSCalibur Flow Cytometer and proportion of CD38+/CD45−/CD138+ cells from MM BM was determined. To isolate MM cells, CD138 microbeads was employed using manufacturers guidelines (Miltenyi Biotec). Cells were washed in beads buffer (PBS/2 mM EDTA/0.5% BSA) and stained with microbeads for 20 mins. CD138+ cells were selected through magnetic isolation using an MS-column (Miltenyi Biotec). DNA extraction was performed using QIAGEN Blood DNeasy Kit (QIAGEN) and quantified with QUBIT Fluorometer 2.0.

OnTarget™ Mutation Detection (OMD) Platform

Genomic DNA from CD138 MM cells and paired plasma (2 mls) from patients was utilised for mutational characterisation with the OnTarget™ Mutation Detection (OMD) platform (Boreal Genomics) that includes 96-mutations in the KRAS, NRAS, CTNNB1, EGFR, PIK3CA, TP53, FOXL2, GNAS and BRAF genes with 43 mutations (BRAF n=6; KRAS n=18; NRAS n=10 and TP53 n=9) potentially relevant to MM (FIGS. 10 and 11). The methods for OMD sample extraction, quantification, processing, mutation enrichment, MiSeq library preparation and sequencing, data analysis are provided below and are as described previously (Kidess E, Heirich K, Wiggin M, Vysotskaia V, Visser B C, Marziali A, et al. Mutation profiling of tumor DNA from plasma and tumor tissue of colorectal cancer patients with a novel, high-sensitivity multiplexed mutation detection platform. Oncotarget. 2015; 6:2549-61).

OnTarqet™ Mutation Detection (OMD) Platform Sample Extraction, Quantification and Processing

DNA from plasma samples was extracted using a modified version of the QIAamp Circulating Nucleic Acid kit (Qiagen, part number 55114). Samples were eluted into a final volume of 60 μl 0.1×TE. DNA samples derived from bone marrow were diluted to 60 pin 0.1×TE. A 5 μl aliquot was removed for quality control. The remainder was kept for use in the OnTarget assay. The number of genome copies present in each DNA sample was assayed using qPCR. The 5 μl sample aliquots were serially diluted 15-fold and 60-fold, and assayed in duplicate by qPCR using amplicons contained within the COG5 and ALB genes. Samples found to contain less than 300 ng, the input mass limit for the assay, were consumed to run the assay while samples containing more than 300 ng were diluted with dH₂O, such that a 50 μl aliquot would yield 300 ng as measured by qPCR. Six (6) negative control samples, containing 30 to 300 ng wild type DNA (Roche Human Genomic DNA, part number 11691112001), were run in parallel to the samples received. Internal positive controls, used to calculate the process yield for each mutation individually and to verify assay success for each mutation, were then added to each sample. The internal positive controls have identical sequence to the mutant alleles at PCR primer and OnTarget homology sites, but additionally contain random identifiers (RIDs), random DNA barcodes which facilitate yield calculations for individual input molecules and allow controls to be easily distinguished from true mutants following sequencing. Approximately 100 internal positive control molecules were added for each mutation in the 96-mutation panel. Each sample was then assigned a unique sample DNA barcode in a multiplex 12Vcycle barcoding PCR reaction (PCR1). 99% of each sample was used as template in a barcoding reaction to amplify the loci containing the 96 mutations in the panel. The remaining 1% was barcoded in a separate reaction in which mutation panel loci and two additional control loci (COG5 and ALB, used in quantification) were amplified. In both barcoding PCR reactions, all primers contain 5′ tags used as universal primers, allowing amplification of all loci with a single primer set in later steps. The barcoded amplified products and quantification reaction products were then pooled, yielding a single aliquot for each sample containing all PCR product for that sample. 15% of the PCR product for each sample was pooled and subsequently purified with using a Zymo DNA Clean and Concentrator column according to the manufacturer's instructions. The remainder of each sample was retained.

OnTarget Mutation Enrichment

The sample sets were loaded into the OnTarget, and enriched for 96 mutations, as well as wild type COG5 and ALB sequences. The enriched OnTarget outputs were then purified using a BioRad Micro BioVSpin 6 column according to the manufacturer's instructions.

MiSeq Library Preparation & Sequencing

Enriched and purified OnTarget outputs were then used for Illumina MiSeq library preparation. Products were amplified and tagged with MiSeq adaptors by 35 cycles of PCR using the universal primers (PCR2), which contain set-specific barcodes and 5′ MiSeq adaptor tags. The PCR output was then purified using the Agencourt AMPure XP kit. The sequencing library was then quantified by qPCR using the KAPA Library Quant kit, normalized to a concentration of 5 nM, and the library was then sequenced on the IIlumina MiSeq.

Data Analysis Sequence Alignment

Sequencing data was analyzed in a fully automated fashion using custom analysis scripts written using BWA for alignment to a custom reference library made up of sequences from within the OnTarget 96-plex mutation panel and SAM Tools for further data manipulation following alignment. Mutation quantification, quality control, and visualization were performed using scripts written in Perl, Python, and MySql and with tools such as Graphviz. A brief description of the algorithm follows. Raw FastQ files from the MiSeq were first de-multiplexed by sample and set barcodes (added in the first and second PCR reactions, respectively), trimmed to retain only the endogenous regions of each molecule lying between the barcoding PCR primers, and then filtered according to the following criteria:

a) Forward and reverse reads must align to the same reference sequence b) Both reads carry the same mutation c) The mutation identified must be contained within the OnTarget 96-plex panel.

Reads satisfying the above conditions were binned according to sample barcode and mutation. The remaining reads were then re-analyzed to determine whether they aligned to a separate reference library for the internal positive control molecules as follows:

1. The first 15 bases of the endogenous section of each read were aligned to a reduced set of reference sequences for the loci within the OnTarget 96-plex panel
2. RID barcodes were found by searching for flanking sequence specific to its locus
3. RID sequences were then removed from the endogenous sequence; the remaining endogenous sequence was then passed through the tests (a)-(c) above.

Internal positive control reads passing the filter were corrected for sequencing errors within the RIDs and binned according to sample barcode, mutation, and unique RID sequence. The average single molecule yield through the entire workflow for each sample barcode/mutation combination was then calculated as the average number of reads over all RIDs for that barcode and mutation.

Quality Control Checks

Quality control checks were performed to ensure that the sequencer detected every IPC molecule, and by extension, every genomic mutant molecule entering the workflow. For each mutation in each sample, two checks were performed:

(a) The number of internal positive control molecules detected must be at least 50% of the expected number of input internal positive control molecules.

(b) The average number of reads observed for each input molecule must be greater than 10. Mutations not conforming to both of the above conditions were flagged as having degraded sensitivity.

Limit of Detection Calculation and Mutation Calling

The number of input mutant molecules for each mutation within each sample was then calculated by dividing the number of mutant reads for a given barcode by the average single molecule yield for that mutation and barcode. A similar process was followed for the WT COG5 and ALB sequences, and used to measure the total number of genomes that entered the workflow; taking into account that only 1% of these loci was amplified in the barcoding PCR reaction. Mutation abundances were calculated as the ratio of input mutant copies to total input genome copies. For two samples, no internal positive controls were added to the samples, which meant it was not possible to directly measure single molecule yield. Single molecule yield for these two samples was approximated by averaging the single molecule yield for the 9 other samples processed in parallel. This approximate single molecule yield was then used to determine mutant copy number from the number of mutant reads. To check the validity of this analysis, this calculation method was performed for all other samples within the set that did contain internal positive controls, and checked against the standard calculation; results differed by less than 20% for all mutations in all samples. Mutations were called as positive only if they detected number of mutant copies was greater than the limit of detection, which is defined as:

$$L O D = 2 cp + \frac{WT\_background}{{N}_{genomes}} + 1 \% * {\sum }_{amplicon} {cp}_{i n}$$

Where the terms in the sum are:
Two input mutant copies
The maximum expected mutant background in WT samples (99.9% confidence interval, 1 tailed), calculated as averge mutation abundance detected in historical Boreal Genomics wildVtype samples plus 3 standard deviations (WT_background), converted to copies by dividing by the number of input genomes (Ngenomes).
The maximum expected crosstalk arising from sequencing errors on other sequences within the amplicon. This is calculated as 1% of the sum all detected input copies (cpᵢₙ) not matching the mutation within the amplicon, and includes both WT and mutations in the 96-plex panel. In cases where one mutation is present at high abundance, this can have a significant impact on the limit of detection for closely related mutations.

Whole Exome Sequencing

For WES, genomic DNA and ctDNA from paired patients, library prep and exome capture were undertaken with the NEBNext Ultra Library prep kit (Genesearch) and SureSelect XT2 human exome V5.0 kit (Agilent), respectively. Sequencing was then undertaken on an Illumina HiSeq 2500 and processed via the APF human exome pipeline.

Droplet Digital PCR (ddPCR): Validation of OMD and Patient ctDNA Tracking

The OMD findings were validated with mutation specific ddPCR and serial PL samples from patients were also quantitatively tracked with ddPCR (Biorad QX200 droplet digital PCR system). PCR was performed using the QX200 ddPCR (Biorad). Droplets were generated using the droplet generator in which the 20 ul reaction is partitioned into an emulsion of up to 20,000 stable nanoliter droplets. The droplets were then subject to PCR amplifications performed using the Prime PCR assay conditions (Biorad). All ddPCR set up had no-template controls. Following PCR, the droplets were read with a two-fluorescence detector to determine droplets that are positive for the mutation of interest. Quantasoft™ software version 1.7 enabled the determination of the mutant copies and fractional abundance (FA) of the samples.

### Example 2

Cell-Free DNA Amounts in MM Patients is Significantly Higher than in Normal Volunteers

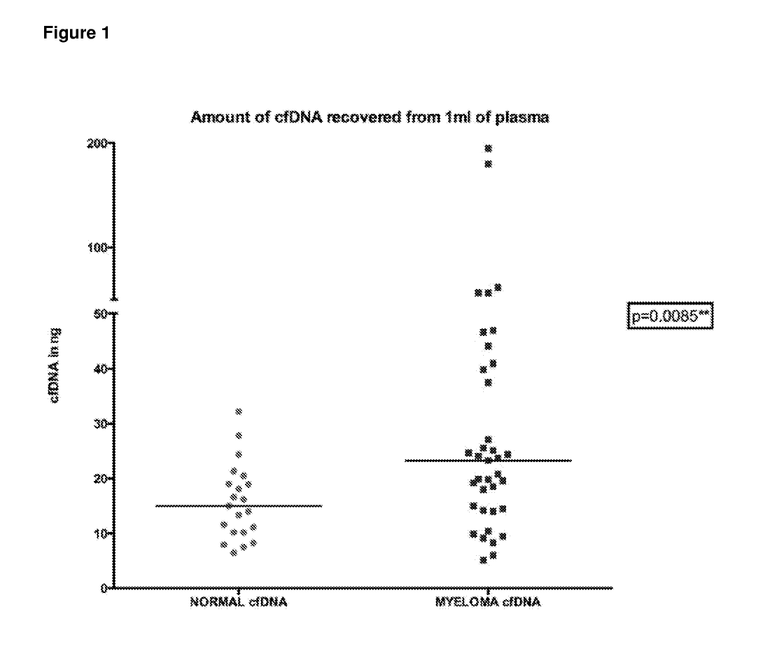

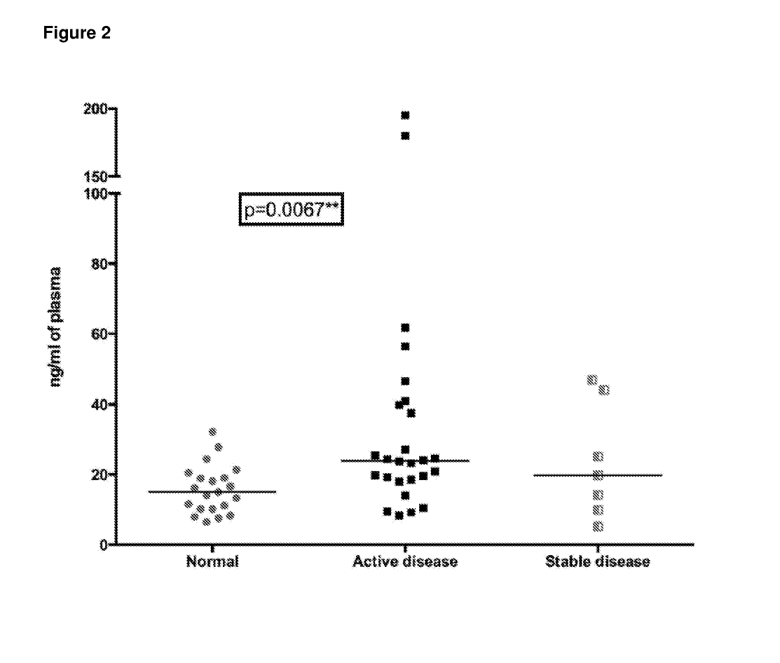

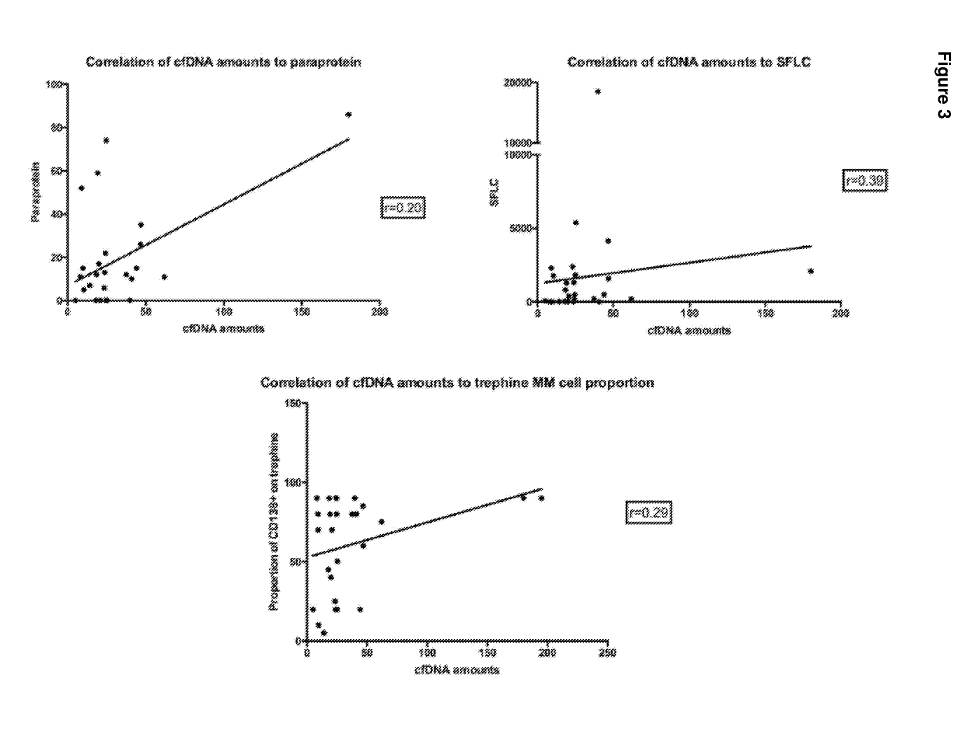

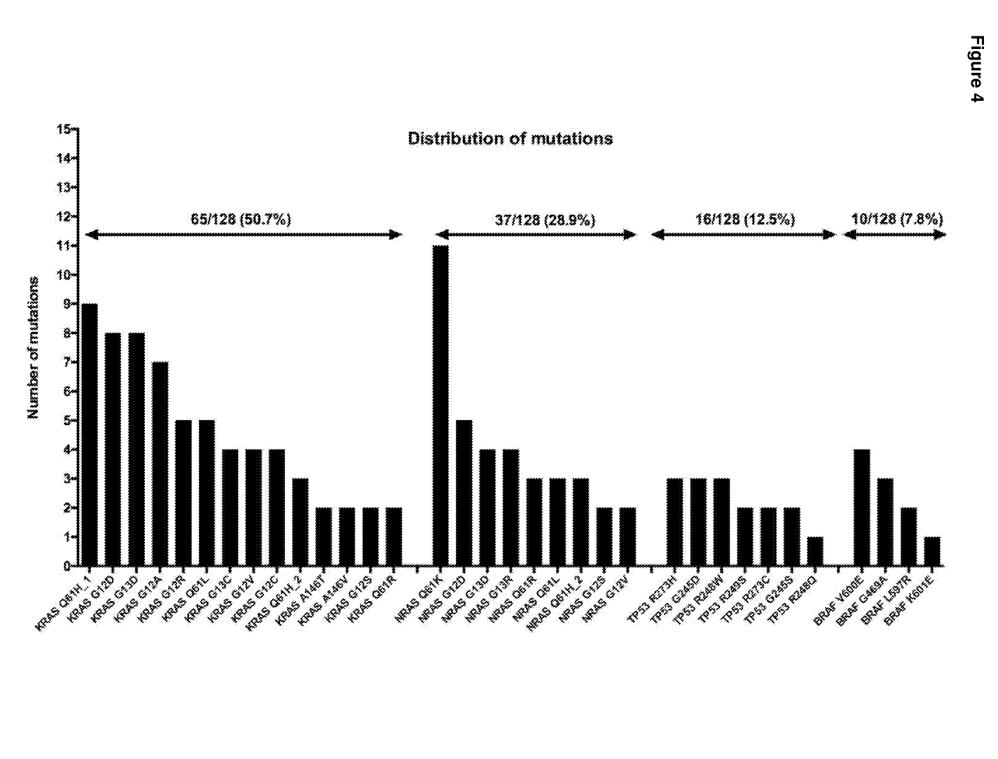

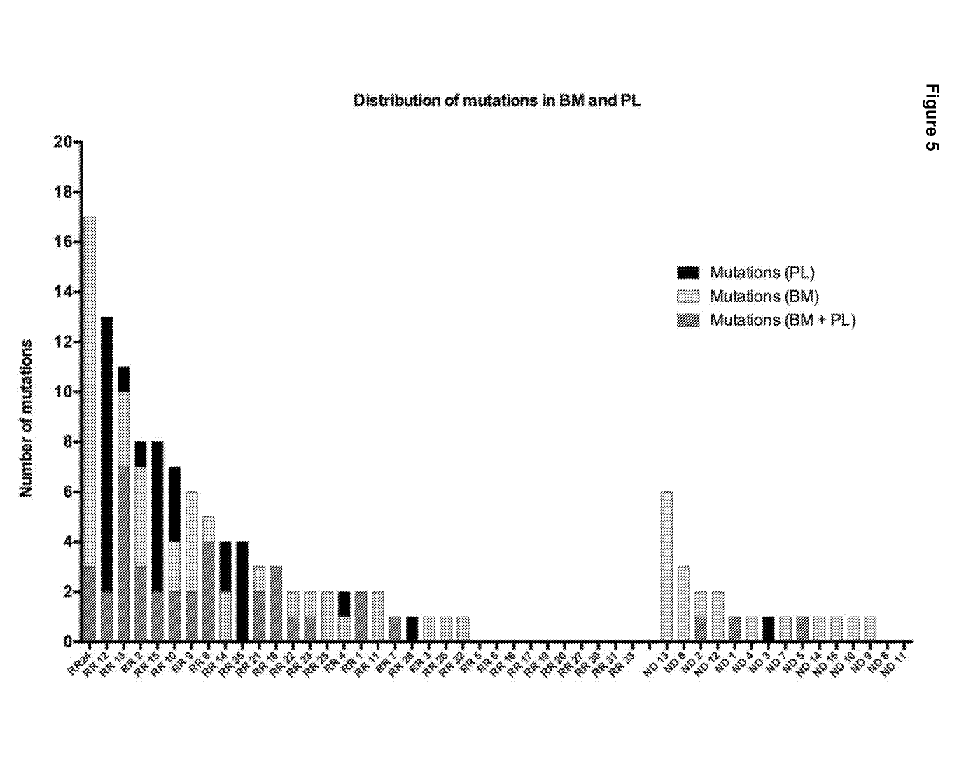

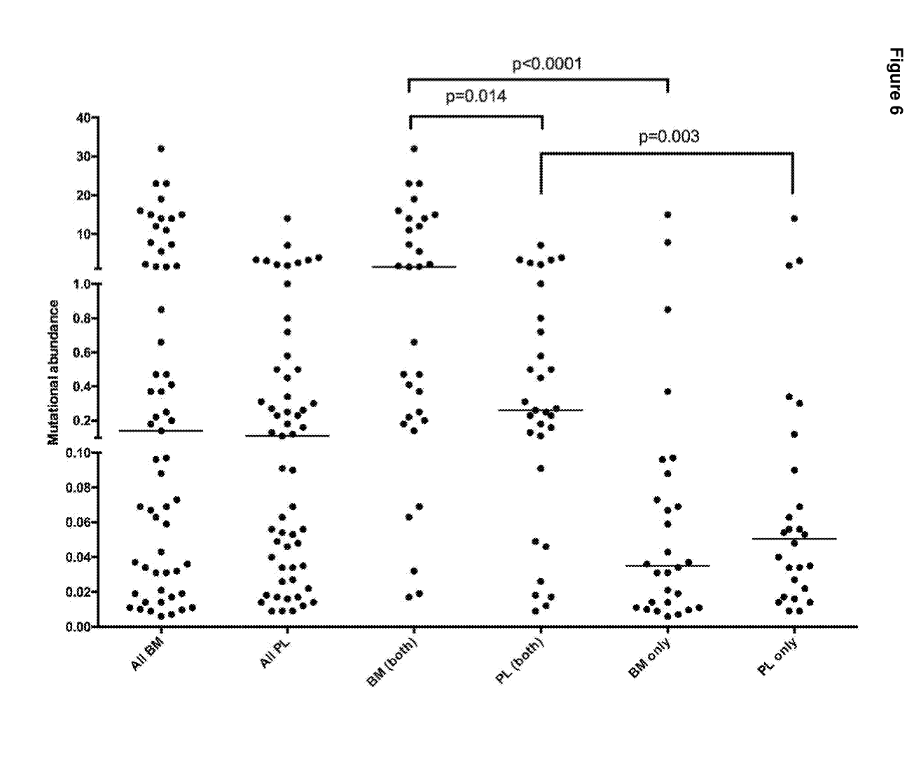

CfDNA amounts in MM patients (n=37) and NV (n=21) were determined. Higher quantities of cfDNA were obtained from MM pts than NV (median 23 ng/ml [range 5-195 ng/ml] versus 15 ng/ml [range 6-32 ng/ml], respectively, p=0.0085, FIG. 1). When the amount of cfDNA was correlated to the disease stage, it was clear that patients with active disease (ND and relapsed disease) had significantly higher amounts of cfDNA compared to NV (p=0.0067; FIG. 2). While the amounts of cfDNA were significantly higher in patients with active disease, the levels had no correlation with amounts of paraprotein, serum free light chains and BM MM cell proportions (FIG. 3, Spearman's rank correlation coefficient).

### Example 3

Profiling of Both BM MM Cells and ctDNA Provides a Comprehensive Picture of the Mutational Landscape of MM Patients

Forty-eight MM pts (15 newly diagnosed [ND] and 33 relapsed/refractory [RR]) had contemporaneous CD138 enriched MM tumour cell populations collected and all paired BM MM DNA and ctDNA specimens along with 6 wild-type (WT) DNA controls underwent OMD. A total of 128 mutations (KRAS n=65 [50.7%], NRAS n=37 [28.9%], BRAF n=10 [7.8%], TP53 n=16 [12.5%]) were detected in the MM patients (BM and/or ctDNA) with none detected in WT controls (FIG. 4). Out of the 128 mutations, n=38 mutations were found both in BM and PL, n=59 in BM and n=31 in PL. Moreover, a total of 53.9% of mutations were found in the PL signifying the existence of ctDNA in MM. Ten of the 48 patients had 31 mutations in the PL not present in the BM, thus a total of 24.2% were detected exclusively, or predominantly, distant to the BM biopsy site (FIG. 10).

Of 48 patients, 12 patients did not have any detectable mutations in either BM or PL (25%), 16 (33.3%) harboured no mutations in the PL and 3 (6%) had mutations only in the PL.

Within the RR cohort, there were more mutations only found in the PL (30 mutations) compared to the ND cohort (1 mutation) consistent with a greater likelihood for the presence of genetically heterogeneous dominant sub-clones in RR patients present exclusively distant to the BM biopsy site (FIG. 5). In addition, a higher frequency of PL-only mutations was detected in RR patients than ND (27.2% vs 6.6%, p=0.25 Chi-squared test). The presence/number of mutations did not have any correlation with the presence of high-risk cytogenetics.

Patients with no mutations detected in either BM or PL (12 patients) were excluded from the validation analysis. The remaining 36 patients from the initial cohort with matched BM and PL were validated for selected mutations using ddPCR. BM and PI samples were tested for 123 mutations by ddPCR with 92.6% concordance between OMD and ddPCR.

Droplet digital PCR (ddPCR) was utilised for subsequent validation of mutations detected in the OMD. A total of 12 patients from the initial cohort with matched BM and PL were validated for selected mutations (KRAS G13D, G12D, G12V, G12A and G12R) using ddPCR. 10/11 (90.9% concordance) mutations that were present by OMD were detected by ddPCR and 4/13 (30.7%) mutations that were negative in the OMD were detected by ddPCR, indicating a higher sensitivity for ddPCR (FIG. 14).

Mutational Abundances are Reflective of the Genetically Heterogenous Landscape of MM Patients

The mutational abundances (MA) of the mutations in the BM ranged from 0.0059%-32% (median 0.14%), and for PL from 0.0090%-14% (median 0.11%) (FIG. 6). The median MA in BM was significantly higher than the median MA of PL for mutations that were found in both BM and PL than for mutations found in BM alone (FIG. 6; p<0.0001), indicating that the more dominant clones in the BM are also detectable in the PL (FIG. 6, p=0.014). This is consistent with the notion that the mutational profile represented in OMD did not include smaller BM clones below the level of detection of the assay. Likewise, the MA of PL only mutations was significantly lower than MA of PL mutations detected in both BM and PL (FIG. 6; p=0.003).

Mutations were Predominantly Associated with the RAS-MAPK Pathway

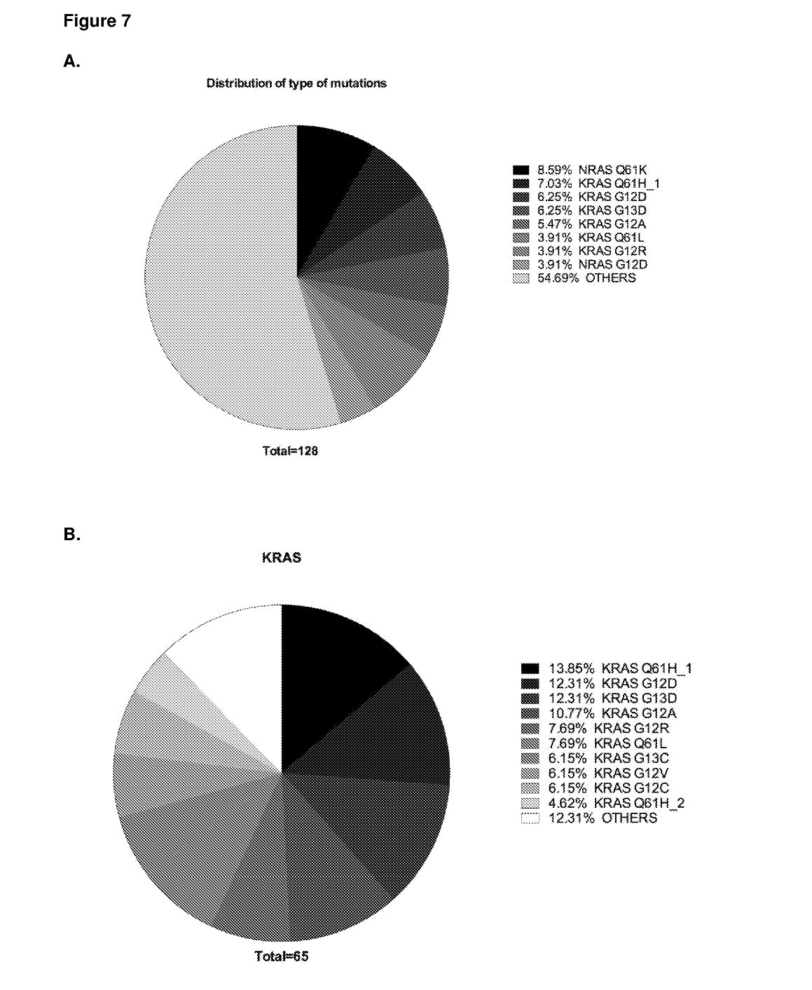

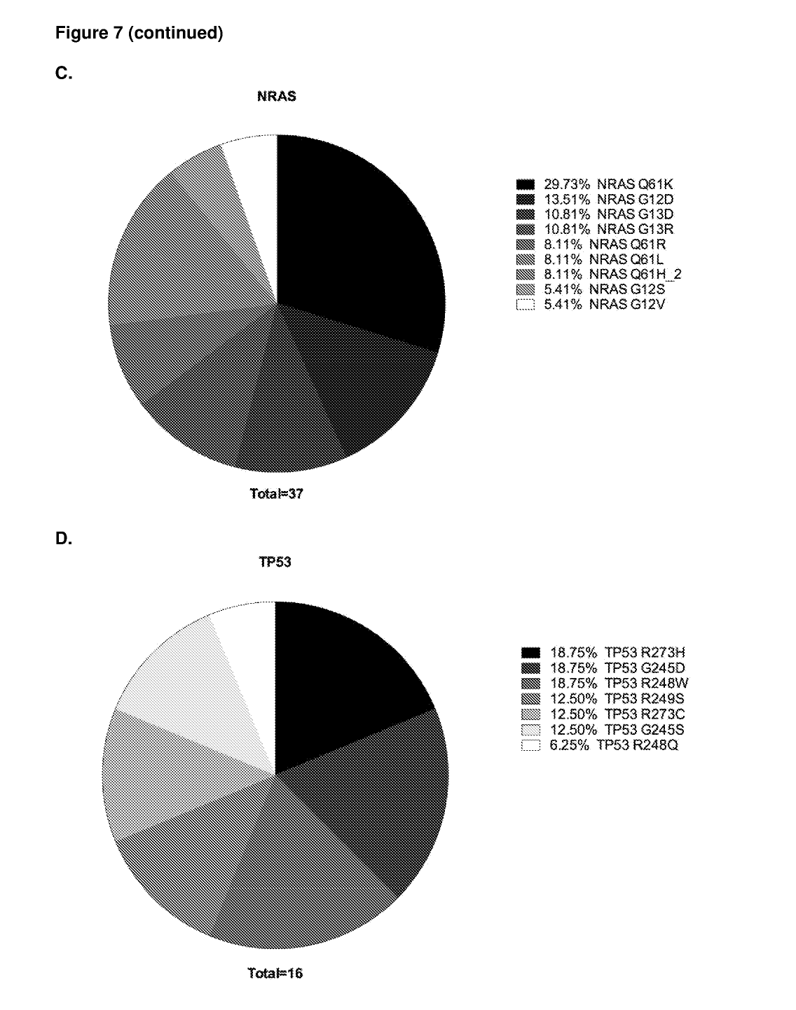

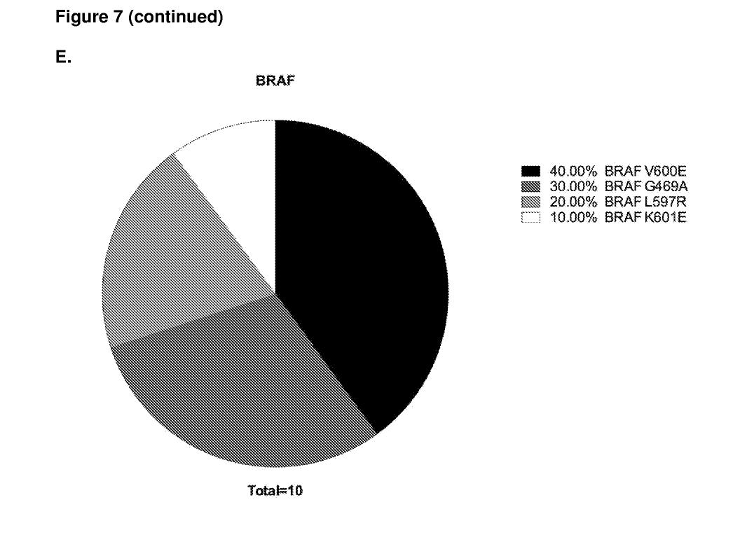

The top 4 mutations found in both BM and PL were NRAS Q61K (8.6%), KRAS Q61H_1 (7.0%), KRAS G13D (6.3%) and KRAS G12D (6.3%) (FIG. 7A). Within the KRAS mutations, KRAS Q61H_1 (13.9%) was the most prominent followed by KRAS G13D (12.3%) and KRAS G12D (12.3%) (FIG. 7B), while in the NRAS mutations, NRAS Q61K (29.7%) had the highest incidence followed by NRAS G12D (13.5%) and NRAS G13D (10.8%) (FIG. 7C). Amongst TP53, TP53 G245D, R273H and R248W had the same incidence (18.8%; FIG. 7D) while BRAF V600E (50%; FIG. 7E) had the highest incidence of all 4 BRAF mutations tested.

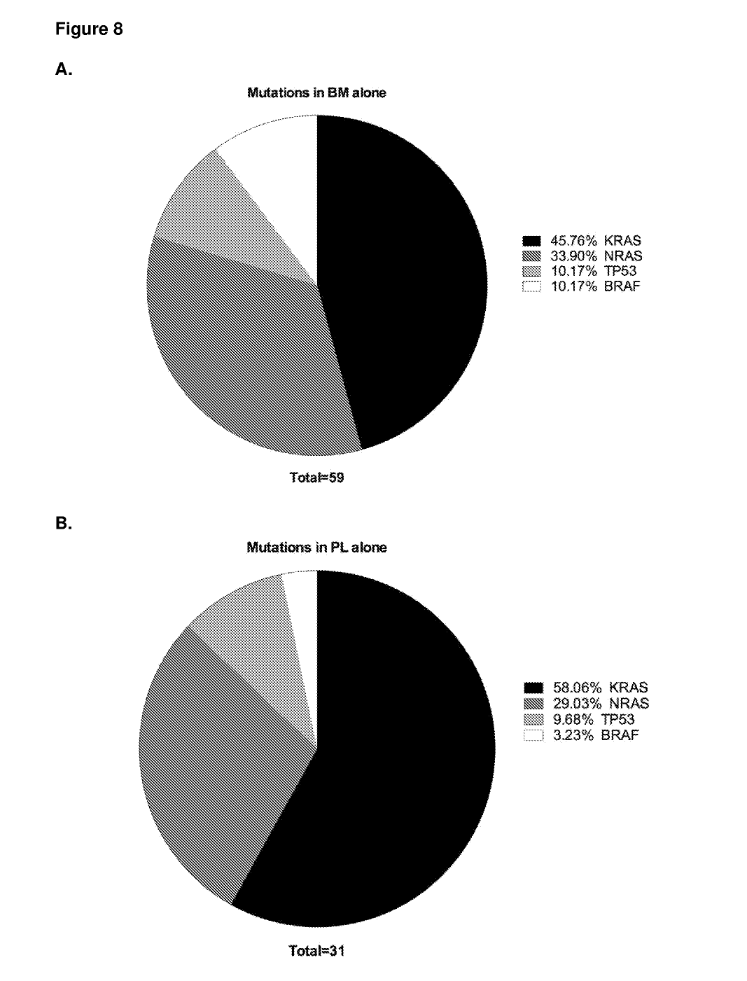

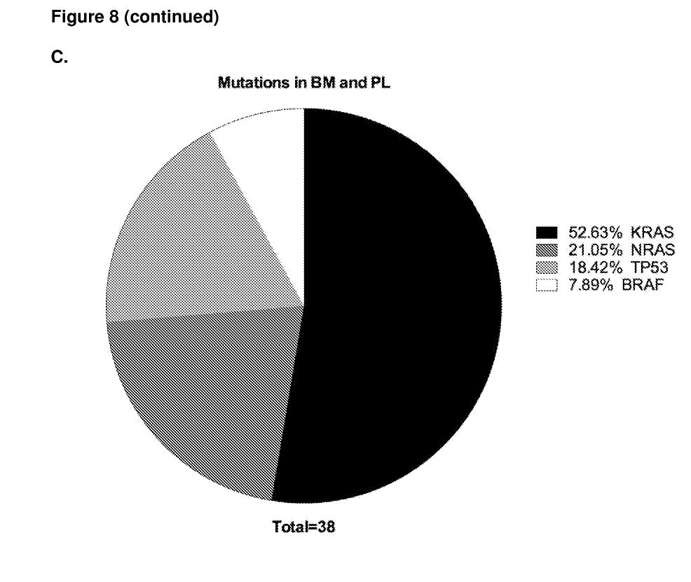

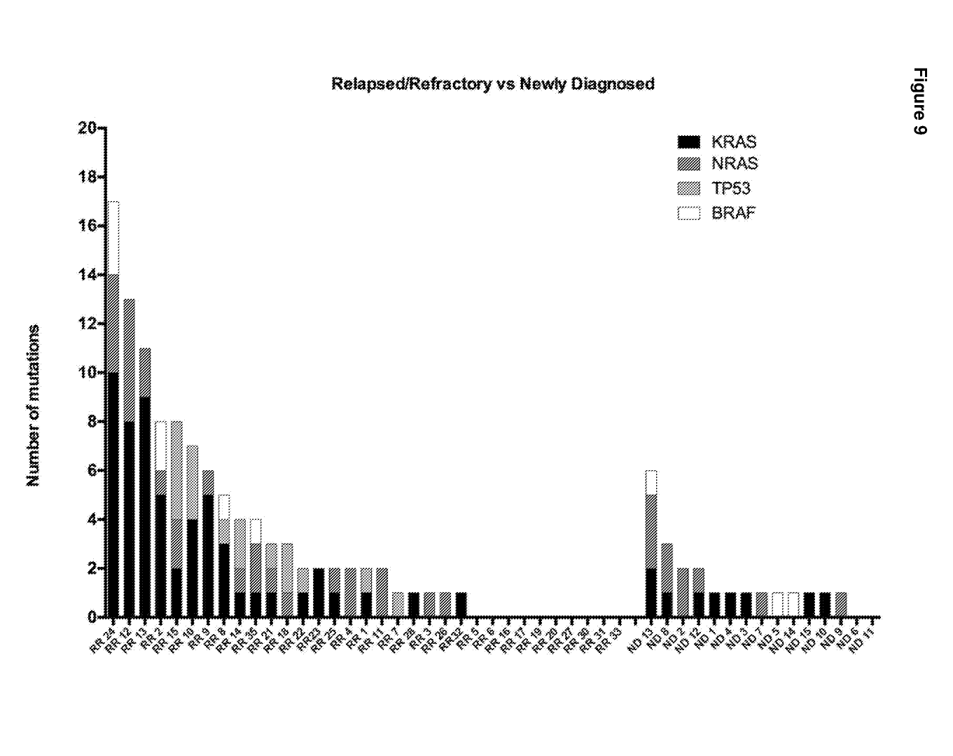

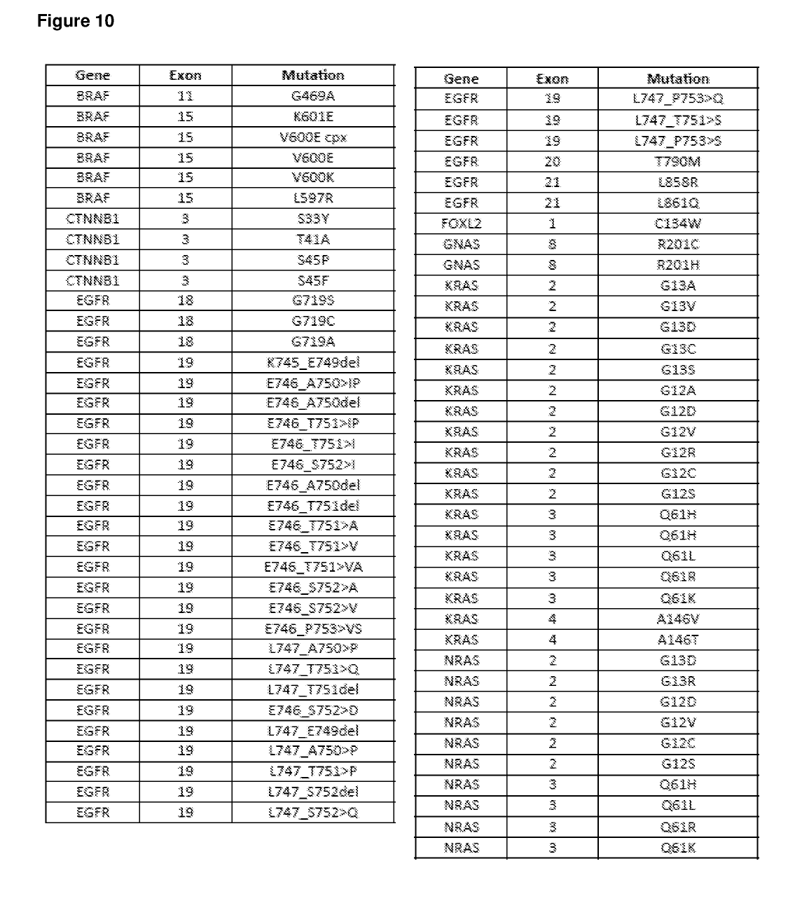

Of the 48 patients tested, 33 (69%) had at least one RAS activating mutation. KRAS mutations had the highest incidence in PL-only, BM-only and both (FIG. 8A-C), (FIG. 8C). RAS mutations were distributed amongst both ND and RR patients, with 73% of ND and 67% of RR patients having of at least one RAS activating mutation (FIG. 9). However, individual patients with advanced disease had higher numbers of RAS mutations, with 15 of 35 (43%) harbouring activating mutations compared to ND, which had 4/15 (27%) (FIG. 9, p=0.35 Chi-squared test). Within the RR cohort, 3 patients (RR24, RR12 and RR13, FIG. 9) had more than 10 activating RAS mutations. Amongst the RAS-MAPK pathway mutations, KRAS had the highest incidence in RR patients (56 mutations), followed by NRAS mutations (27 mutations). However, in ND patients, NRAS (10 mutations) were higher than KRAS (9 mutations). These results indicate that KRAS and NRAS mutations are predominant in MM. While RAS-MAPK pathway mutations were high in incidence, TP53 mutations were found exclusively in RR patients (FIG. 9).

TABLE 1
Sample information. Table has information about samples and the
mutational profile of the samples.

| | |
|---|---|
| No. of samples | 48 |
| Newly diagnosed:Relapsed | 15:33 |
| Total no: of mutations | 128 |
| Mutation proportions | KRAS (65/128; 50.7%) |
| | NRAS (37/128; 28.9%) |
| | BRAF (10/128; 7.8%) |
| | TP53 (146128; 12.5%) |
| Mutations in both BM and ctDNA | 38 |
| Mutations in only BM | 59 |
| Mutations in only ctDNA | 31 |

### Example 4

MM Patients with Advanced Disease Harbour More Mutations in ctDNA

More MTS were present in RR pts compared with ND pts—median 2.5 mutations/patient (range, 0-11) versus 1 mutations/patient (range, 0-3), respectively, p=0.03. Activating MTS of the RAS-MAPK pathway (KRAS/NRAS/BRAF) were detected (BM and/or ctDNA) in 22 of 28 pts (79%) comprising 90% of ND pts (median MTS 1, range 0-3) and 72% of RR pts (median MTS 1, range 0-11), moreover, 8 of 18 (44%) RR pts harboured activating MTS (2, 2, 3, 4, 4, 8, 8, 11 each). In addition, all 13 TP53 MTS were found exclusively in RR patients.

### Example 5

Whole Exome Sequencing can be Utilised for ctDNA Mutation Detection

Exploratory WES was undertaken on 4 ctDNA samples and demonstrated predominantly exonic variants of 108, 152, 101 and 98 distinct genes with median read depths of 115, 79, 78 and 65, respectively. Variants were enriched for C>T transitions (51%, 45%, 51% and 44% of all variants, respectively) reflecting spontaneous deamination of methylated cytosine to thymine as has been described with WES of MM BM.

The data herein confirm the utility of ctDNA evaluation as an adjunct to the mutational characterization of MM. Furthermore, using highly sensitive targeted approaches it has been demonstrated a more complex mutational landscape in MM than previously shown with BM WES. In the cohort herein, activating MTS of the RAS-MAPK pathway were highly prevalent with the findings suggesting a striking subclonal convergence on this pathway. The high-sensitivity approaches incorporating plasma ctDNA evaluation aimed at identifying actionable MTS may represent a significant advance in attempts to personalize future MM treatment strategies and that future studies incorporating RAS-MAPK pathway targeted approaches for MM are essential.

### Example 6

Methodology for the 7 Patients Monitored for Mutation Transcripts in Following Examples

Patients were provided with written informed consent for blood to be collected for the research study which was approved by the Alfred Hospital Human Research Ethics Committee, Melbourne, Australia.

Blood samples were obtained from patients based on clinical requirements. All blood samples were collected in Streck DNA tubes and were processed within 48 hours after collection. Plasma was isolated after centrifugation at 800 g for 10 minutes followed by a further 10 minutes centrifugation at 1600 g before plasma was aliquoted in 1.8 ml tubes and stored at −80° C. till required. Plasma DNA was extracted using the QIAamp Circulating Nucleic Acid Kit (Qiagen) as per manufacturer's instructions. DNA was eluted using 100 μl of Buffer AVE and quantified using NanoDrop 1000 Spectrophotometer (ThermoScientific).

Mutations in each patient were identified by Boreal Genomics and we tracked and quantitated the mutations using droplet digital polymerase chain reaction (DDPCR) (BioRad). We used the BioRad QX200 DDPCR system and DDPCR probe mastermix [DDPCR Supermix for probles (no dUTP)] and primers which target the specific mutant clones. These mutant and wild type primers were purchased from BioRad and these primer sequences are proprietary to the company.

The DNA samples were fragmented by restriction enzyme digest which is achieved by direct addition of MSE1 to the DDPCR reaction at a concentration of 5 units per 20 μl of DDPCR reaction and ran on a C1000 Touch Thermal Cycler with 96-Deep well reaction module. The thermal cycling protocol for the amplification of mutations is 95° C. for 10 minutes to activate the enzyme followed by denaturation at 94° C. for 30 seconds. Annealing was at 55° C. for 60 seconds and the denaturation and annealing steps were repeated 39× followed by enzyme deactivation at 98° C. for 10 minutes and the reactions were held at 12° C. till samples are removed from the machine. The ramping rate for all the steps were set at 2° C. per second. After completion of the PCR steps, samples were then loaded onto the QX200 Droplet Reader and analysed using QuantaSoft Ver 1.7.

### Example 7

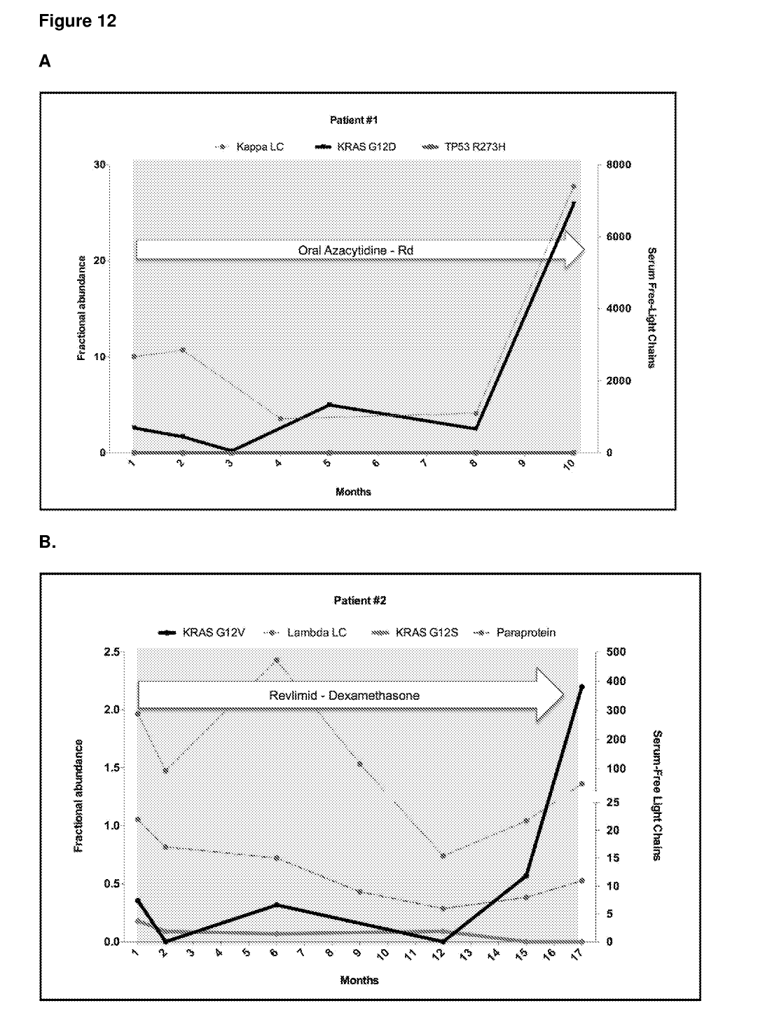

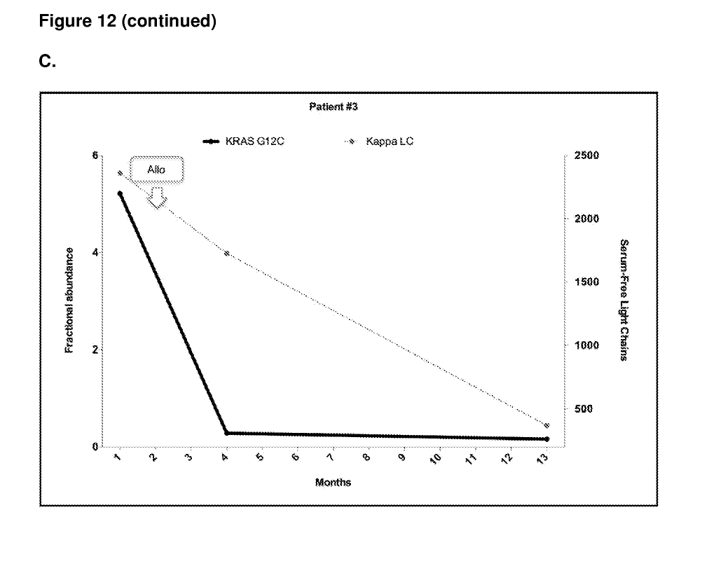

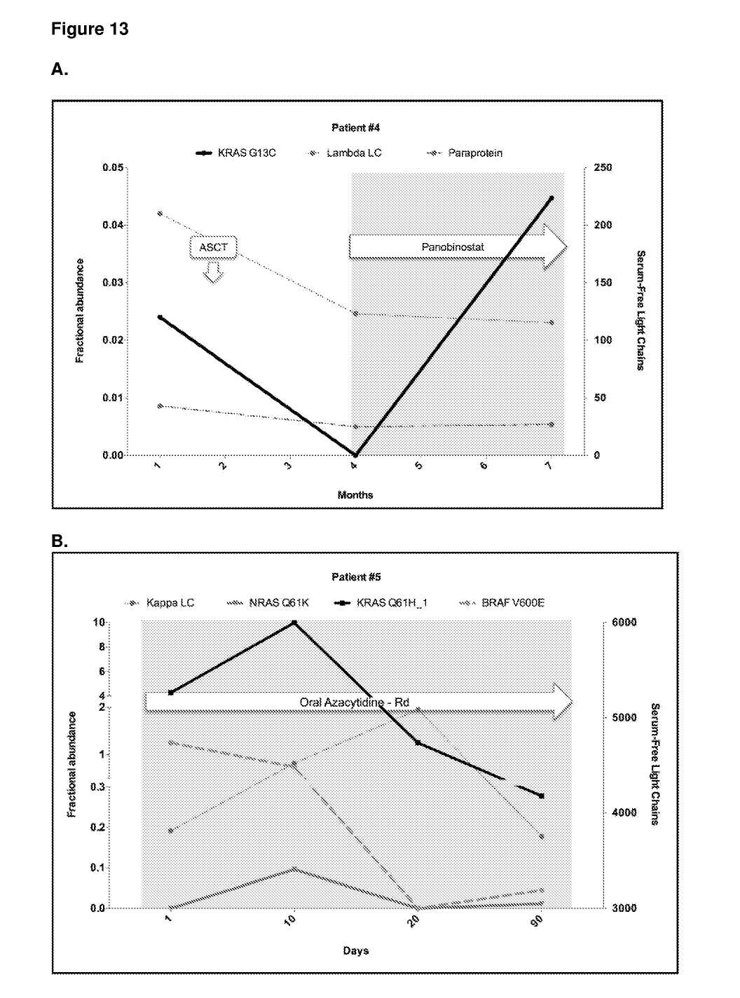

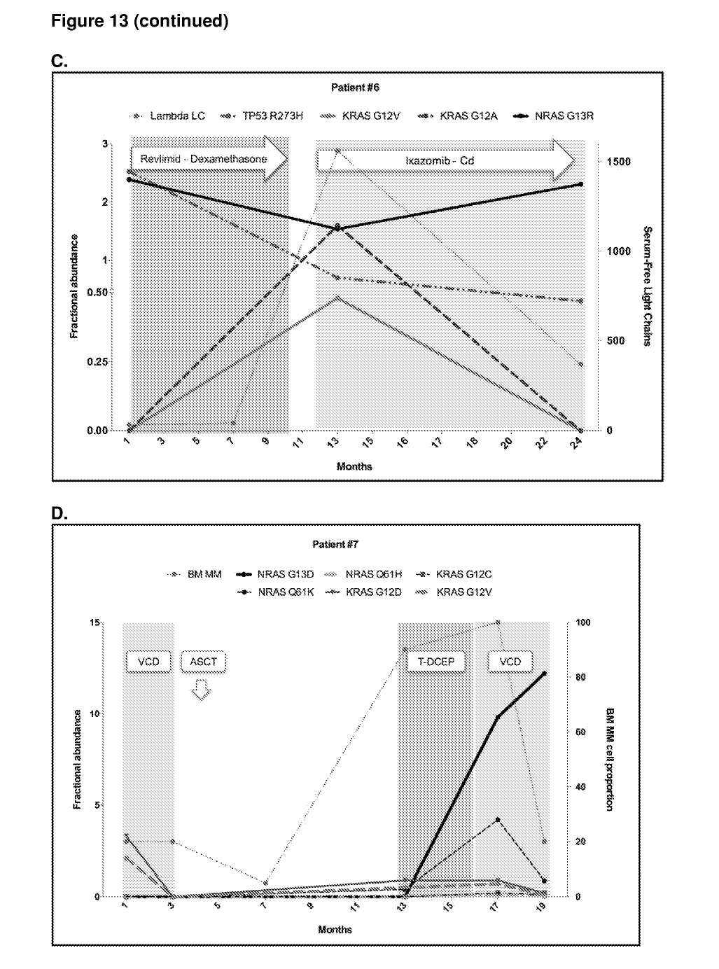

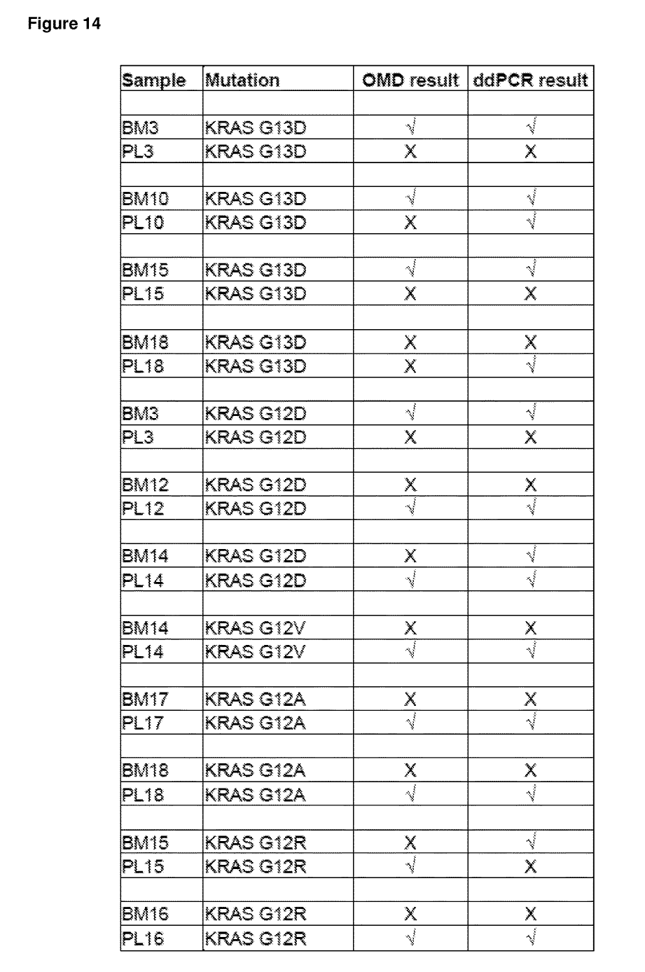

Patient #1 with advanced relapsed disease had sequential PL samples collected while being treated on a phase 1 b trial of oral azacytidine combined with revlimid and dexamethasone (Rd). ctDNA was tested by ddPCR for previously identified TP53 R273H and KRAS G12D mutations. The FA of KRAS G12D, which appeared to be the dominant clone, rapidly increased coincident with relapse of the disease, while that of TP53 273H did not change significantly over time (FIG. 12A).

This clinical example in a human patient suffering from multiple myeloma (specifically a IgG lambda form) shows the heterogeneity of mutations that are detectable at different stages of disease.

### Example 8

Patient #2 had relapsed MM and had sequential PL samples collected while on with revlimid and dexamethasone (Rd). Analysis of ctDNA for two mutations, KRAS G12S and KRAS G12V indicated that the KRAS G12S mutation exhibited minimal change over time, while the FA of the KRAS G12V clone changed in parallel with changes in levels of lambda light chains (LC), both increasing with emergent refractoriness to therapy (FIG. 12B).

This clinical example in a human patient suffering from multiple myeloma (specifically a IgG lambda form) shows the heterogeneity of mutations that are detectable at different stages of disease.

### Example 9

Patient #3 had relapsed MM with sequential PL collected at the time of relapse and post-allograft. Levels of Kappa LC gradually decreased over a period of 12 months post-allograft; in contrast FA of mutant clone KRAS G12C had a sharp decline post-allograft with only low detectable levels remaining in the PL consistent with stable disease (FIG. 12C).

This clinical example in a human patient suffering from multiple myeloma (specifically a IgG kappa form) shows the decline in serum markers after the decline in circulating tumour DNA.

### Example 10

Patient #4 was a newly diagnosed MM enrolled in a Phase II study of panobinostat for MM patients failing to achieve complete response following high-dose chemotherapy conditioned autologous stem cell transplantation (ASCT). Sequential PL samples pre and post-ASCT and after 3 months of panobinostat treatment were analysed for the presence of KRAS G13C, a PL-only mutation not identified in the BM. The FA of KRAS G13C increased while on therapy with minimal changes in PP or Lambda LC heralding subsequent relapse and cessation of trial therapy (FIG. 13A).

This is a clinical example of ctDNA as better biomarker of disease status with changes in FA either preceding or showing discordance with observable changes in standard measures of tumour burden enabling therapy switch earlier than that predicted by the serum marker.

### Example 11

Patient #5 had advanced relapsed MM with sequential PL collected over a period of 90 days while being treated on a phase 1b trial of oral azacytidine combined with Rd. Three mutant clones, NRAS Q61K, KRAS Q61H_1 and BRAF V600E were tracked with a sharp decline in the FA of all three mutant clones observed within 10 days following treatment. In contrast, Kappa LC continued to rise until day 20. Level of KRAS Q61H_1, which had the highest FA continued to decline till day 90, coinciding with the Kappa LC levels (FIG. 13B).

Clinical example of ctDNA as better biomarker of disease status with changes in FA either preceding or showing discordance with observable changes in standard measures of tumour burden enabling prediction of disease response earlier than serum markers.

### Example 12

Patient #6 had relapsed disease with reduction in both the TP53 R273H and NRAS G13R clones on Rd. A rapid increase in Lambda LC consistent with light-chain escape was coincident with the emergence of two new KRAS clones, G12A and G12V in the PL that were not previously detected. The FA of both KRAS clones reduced with switching to Ixazomib-cyclophosphamide-dexamethasone (ICd) therapy coinciding with a serological response. However, FA of the NRAS G13R clone that was responsive to Rd elevated on ICd in contrast to the TP53 R273H clone that continued to respond, highlighting the differential responses of the 4 mutant clones to two different lines of therapy (FIG. 13C).

Clinical example of ctDNA as better biomarker of disease status with changes in FA either preceding or showing discordance with observable changes in standard measures of tumour burden enabling prediction of disease response earlier than serum markers and possibly enabling therapy switch. It also shows the rise in new mutations that correlate with refractory disease whereas pre-existing mutations remain at the same level.

### Example 13

Patient #7 had newly diagnosed non-secretory MM with no conventional peripheral blood markers. Sequential PL ctDNA analysis showed that relapsed disease was associated with the reappearance of mutant KRAS G12V and KRAS G12D clones that had been present at diagnosis in the BM and the emergence of two new clones, NRAS G13D and NRAS Q61K, with the former showing refractoriness to both Thalidomide—dexamethasone, cyclophosphamide, etoposide and cisplatin (T-DCEP) and re-treatment with bortezomib (velcade)—cyclophosphamide—dexamethasone (VCD) and persisting until the patient died shortly thereafter from progressive disease (FIG. 13D). Interestingly the BM biopsy at month 19 showed apparent reduction in disease burden coincident with reintroduction of VCD but ddPCR of PL ctDNA showed an increasing FA of the NRAS G13D clone consistent with VCD-refractory disease.

This clinical example in a human patient suffering from multiple myeloma highlights the clinical utility of detecting circulating cell-free DNA for mutations as a marker of disease state and treatment efficacy. This example clearly shows that patients where no conventional peripheral blood biomarker (i.e. no paraprotein) would particularly benefit from detecting circulating cell-free DNA levels or mutations.

The clinical results in the examples described herein clearly show the benefit of the non-invasive testing of circulating cell-free nucleic acids in patients suffering from various forms of multiple myeloma which can be used to identify stage of disease and treatment efficacy. This enables physicians and medical practitioners to more thoroughly understand the overall mutations status of the disease leading to more tailored treatments that are directed to the signalling pathways driving cancer progression.

Multi-focal tumour deposits and intra-clonal heterogeneity in MM patients provide a difficult setting for comprehensive mutational characterization using WGS or WES at a single BM site, because of its spatial and temporal limitations. A number of secondary activating mutations in RAS, FGFR3, TRAF3 and TP53 are known to be prevalent when the disease relapses, indicating that inclusive characterisation could inform treatment decisions. An alternative approach that could provide a more comprehensive picture of the genetic landscape of individual MM patients is to analyse ctDNA derived from PL, as this theoretically contains a representation of the entire tumour genome that arises from multiple independent tumours. The study described herein in MM sought to evaluate the utility of PL-derived ctDNA as an adjunct to BM biopsy for mutational characterisation and real-time monitoring of mutant clones during patient therapy.

In this study, evaluation of ctDNA in PL and paired BM samples from ND and RR MM patients was performed using the OMD platform to characterise the mutational profile of MM patients focusing on 4 genes of relevance to MM, namely KRAS, NRAS, BRAF and TP53. Mutations were detected both in the BM and PL samples indicating the utility of ctDNA as an accessory to BM biopsy for comprehensive mutational characterisation of MM. The concept of ctDNA being sourced from multiple independent tumours was reinforced with 31% of mutations found only in the PL. In a subset of patients (23%), with mutations found in both BM and ctDNA, the mutational load was proportionately greater within the ctDNA, further strengthening this notion. Additionally, RR patients had a higher incidence of PL only mutations, endorsing the concept that the genetic architecture evolves across multiple tumour sites during disease progression. Likewise, when sequential ddPCR of PL was performed, Patient #1 had an emergent NRAS G13D clone, distant to the BM site, corresponding with refractory disease relapse (FIG. 12). Together, these results provide confirmation that a single-site BM biopsy is limited in its capacity to comprehensively capture the evolving tumour genome, especially if real-time monitoring of the tumour dynamics and predicting resistance to treatment is desired.

In the cohort described herein, activating mutations of the RAS-MAPK pathway were highly prevalent, with at least 1 activating mutation being present in 79% of patients in the BM and/or PL, thus demonstrating a striking sub-clonal convergence on this pathway. These data also confirm that the sub-clonal architecture in some patients is more complex than suggested by prior NGS studies, with a greater prevalence of RAS mutations than previously described, and reinforcing the necessity for appropriately designed prospective clinical trials targeting the RAS-MAPK pathway. Additionally, the use of ddPCR of ctDNA for the quantitative tracking of specific mutant sub-clones should better inform treatment decisions. Moreover, the increase in the number of mutations detected with this approach (compared with WES BM analyses), thus enabling the recognition of patients with a hyper-mutated picture e.g. RR patients 1-4 (FIG. 9), is of therapeutic relevance. Recent observations suggest that check point inhibition is more efficacious in solid tumour patients with the highest mutational loads. Such therapeutic strategies may be brought to bear in a more directed fashion in the context of more comprehensive mutational characterizations of patients.

Similarly, sequential ctDNA analysis using ddPCR of previously identified mutations revealed an increase in FA of specific mutant clones coincident with or preceding relapse (FIGS. 12 and 13). Based on this, it is proposed that targeted ctDNA evaluation for the presence of potentially actionable mutations may provide not only a non-invasive real-time measure of tumour burden but also critical information for therapeutic choice. Moreover, the quantitative data derived from ctDNA evaluation may represent a more holistic measure of whole body tumour burden and subsequent evaluation of response to targeted therapy than that derived from single site BM biopsy examinations. This is the first proof-of-concept study in MM that has evaluated the utility of ctDNA as an adjunct for the mutational characterization of MM. Using highly sensitive targeted approaches the inventors have demonstrated a more complex mutational landscape in MM than previously shown with BM WES and importantly the existence of mutant clones present predominantly or exclusively, distant to the BM biopsy site that can be tracked during patient therapy. The inventors conclude that high-sensitivity approaches incorporating PL ctDNA evaluation aimed at identifying actionable mutations represent a significant advance in attempts to personalize future MM treatment strategies and that future studies incorporating RAS-MAPK pathway targeted approaches for MM are essential.

1. A method for monitoring the response of an individual to treatment for multiple myeloma, the method comprising
providing cell-free nucleic acids derived from a sample of peripheral blood from an individual that has undergone treatment for multiple myeloma;
assessing the cell-free nucleic acids for a mutation in any one or more nucleotide sequences from a KRAS, NRAS, BRAF and/or TP53 gene;
wherein an absence of, or reduction in the number of, mutations in a nucleotide sequence from a KRAS, NRAS, BRAF and/or TP53 gene indicates a response of the individual to treatment for multiple myeloma; or wherein the presence of, or increase in the number of mutations in a nucleotide sequence from a KRAS, NRAS, BRAF and/or TP53 gene indicates a non-response of the individual to treatment for multiple myeloma.

2. A method for monitoring the response of an individual to treatment for multiple myeloma, the method comprising
providing cell-free nucleic acids derived from a sample of peripheral blood from an individual that has undergone treatment for multiple myeloma;
providing nucleic acids from bone marrow mononuclear cells of the individual;
assessing the cell-free nucleic acids for a mutation in any one or more nucleotide sequences from a KRAS, NRAS, BRAF and/or TP53 gene;
assessing the nucleic acids from bone marrow mononuclear cells for a mutation in any one or more nucleotide sequences from a KRAS, NRAS, BRAF and/or TP53 gene;
wherein an absence of, or reduction in the number of, mutations in a nucleotide sequence from a KRAS, NRAS, BRAF and/or TP53 gene in either or both the cell-free nucleic acids or the nucleic acids from bone marrow mononuclear cells indicates a response of the individual to treatment for multiple myeloma; or wherein an presence of, or increase in the number of, mutations in a nucleotide sequence from a KRAS, NRAS, BRAF and/or TP53 gene in either or both the cell-free nucleic acids or the nucleic acids from bone marrow mononuclear cells indicates a non-response of the individual to treatment for multiple myeloma.

3. A method of claim 1 or 2, wherein the mutation detected encodes a mutation selected from the group consisting of those shown in FIG. 10.

4. A method of any one of claims 1 to 3, wherein the method comprises comparing the cell-free nucleic acid from the individual to cell-free nucleic acids obtained from the individual before treatment for multiple myeloma.

5. A method of any one of claims 2 to 4, wherein the method comprises comparing nucleic acids from bone marrow mononuclear cells from the individual to nucleic acids from bone marrow mononuclear cells obtained from the individual before treatment for multiple myeloma.

6. A method of any one of claims 1 to 5, wherein the mutation detected encodes a mutation selected from the group consisting of KRAS G12D, KRAS G12C, KRAS G12V, KRAS G12S, KRAS G12R, KRAS G12A, KRAS G13C, NRAS Q61K, NRAS Q61H_1, NRAS G13D, NRAS Q61H, NRAS Q61L, NRAS G13R, BRAF V600E, and TP53 R273H.

7. A method for determining whether an individual has multiple myeloma, or at risk of developing same, the method comprising:
providing a test sample of peripheral blood from an individual for whom a diagnosis of multiple myeloma is to be determined;
assessing the test sample for the level of cell-free DNA, thereby forming a test sample profile;
providing a control profile containing data on the level of cell-free DNA in peripheral blood of an individual without multiple myeloma;
comparing the test sample profile with the control profile to identify whether there is a difference in the level of cell-free DNA as between the test sample profile and the control profile;
determining that the individual has multiple myeloma, or is at risk of developing same, where the level of cell-free DNA in the test sample profile is higher than the control profile.

8. A method according to claim 7, wherein the step of providing a test sample of peripheral blood includes obtaining a peripheral blood sample directly from the individual to be diagnosed.

9. A method according to claim 7 or 8, wherein the step of assessing the test sample for the level of cell-free DNA includes extracting cell-free DNA from the peripheral blood and discarding all components of the peripheral blood except for the cell-free DNA.

10. A method for diagnosing an individual as having multiple myeloma, or at risk of developing same, the method comprising:
providing a test sample of peripheral blood from an individual for whom a diagnosis of multiple myeloma is to be determined;
assessing the test sample for circulating tumour free nucleic acids,
wherein a detection of circulating tumour free nucleic acids diagnoses that the individual has multiple myeloma, or is at risk of developing same.

11. A method according to claim 10, wherein the circulating tumour free nucleic acids are cell-free nucleic acids in which at least one mutation is present in a nucleotide sequence from a KRAS, NRAS, BRAF and/or TP53 gene.

12. A method according to claim 11, wherein the mutation is any one or more of the mutations listed in FIG. 10.

13. A method for diagnosing an individual as having multiple myeloma, or at risk of developing same, the method comprising:
providing cell-free nucleic acids derived from a sample of peripheral blood from an individual for whom a diagnosis of multiple myeloma is to be determined;
assessing the cell-free nucleic acids for a mutation in any one or more nucleotide sequences from a KRAS, NRAS, BRAF and/or TP53 gene;
wherein detection of a mutation in any one or more of the KRAS, NRAS, BRAF or TP53 diagnoses that the individual has multiple myeloma, or is at risk of developing same.

14. A method according to claim 13, further comprising a step of obtaining a peripheral blood sample from the individual from which cell-free nucleic acids are extracted.

15. A method according to claim 13 or 14, wherein the mutation detected encodes a mutation selected from the group consisting of those shown in FIG. 10.

16. A method according to claim 15, wherein the mutation is selected from KRAS G12D, KRAS G12C, KRAS G12V, KRAS G12S, KRAS G12R, KRAS G12A, KRAS G13C, NRAS Q61K, NRAS Q61H_1, NRAS G13D, NRAS Q61H, NRAS Q61L, NRAS G13R, BRAF V600E, and TP53 R273H.

17. A method for diagnosing an individual as having multiple myeloma, or at risk of developing same, the method comprising:
providing cell-free nucleic acids derived from a sample of peripheral blood from an individual for whom a diagnosis of multiple myeloma is to be determined;
assessing the cell-free nucleic acids for a mutation in any one or more sequences from a KRAS, NRAS, BRAF and/or TP53 gene;
providing nucleic acids from bone marrow mononuclear cells from the individual;
assessing the nucleic acids from bone marrow mononuclear cells for a mutation in any one or more nucleotide sequences from a KRAS, NRAS, BRAF and/or TP53 gene;
wherein detection of mutations in both the cell-free nucleic acids and the nucleic acids from bone marrow diagnoses the individual as having multiple myeloma.

18. A method according to claim 17, wherein the mutations in a nucleotide sequence from a KRAS, NRAS, BRAF and/or TP53 gene are selected from the group consisting of those listed in FIG. 10.

19. A method for diagnosing advanced disease in an individual having multiple myeloma, the method comprising:
providing cell-free nucleic acids derived from a sample of peripheral blood from an individual for whom a diagnosis of advanced disease is to be determined;
assessing the cell-free nucleic acids for one or more mutations in a nucleotide sequence from a TP53 gene;
wherein detection of one or more mutations in TP53 diagnoses the individual as having advanced disease.

20. A method according to claim 19, wherein the mutations in TP53 are any one or more of those listed in FIG. 10.

21. A method for diagnosing a method for diagnosing advanced disease in an individual having multiple myeloma, the method comprising:
providing cell-free nucleic acids derived from a sample of peripheral blood and bone marrow mononuclear cells from an individual for whom a diagnosis of multiple myeloma is to be determined;
assessing the cell-free and bone marrow derived nucleic acids for mutations in any one or more of the KRAS, NRAS, BRAF or TP53;
wherein detection of greater than 3 TP53 mutations diagnoses the individual as having advanced disease.

22. A method according to claim 21, wherein the mutations in TP53 are any one or more of those listed in FIG. 10.

23. A method according to any one of claims 1 to 22, further comprising the step of administering a drug to treat the individual diagnosed as not responding to treatment, having multiple myeloma, active disease or advanced disease.

24. A method according to claim 23, wherein the drug targets the RAS/MAPK pathway.

25. A method according to claim 24, wherein the drug is selected from the group consisting of trametinib, rigosertib, cobimetinib, selumetinib, sorafenib and vemurafenib.

26. A method according to any one of claims 1 to 6, wherein when an assessment is made that the individual is not responding to treatment, the method further comprises the step of replacing or supplementing the existing treatment with additional drugs.

27. A method according to claim 26, wherein the additional drugs are selected from Dexamethasone, Cyclophosphamide, Thalidomide, Lenalinomide, Etopside, Cisplatin, Bortezomib, Cobimetinib, Ixazomib, Rigosertib, Selumetinib, Sorafenib Trametinib, Vemurafinib, Panobinostat, Azacytidine, Pembrolizumab, Nivolumumab, Durvalumab or autologous stem cell transplant (ASCT).
